(19) United States
(12) Patent Application Publication
(10) Pub. No.: US 2020/0232979 A1
(43) Pub. Date: Jul. 23, 2020

(54) Microcapsules and Methods for Analyte Detection

(71) Applicant: Mayo Foundation for Medical Education and Research, Rochester, MN (US)

(72) Inventors: Alexander Revzin, Rochester, MN (US); Ali Rahimian Mashhadi, Rochester, MN (US)

(73) Assignee: Mayo Foundation for Medical Education and Research, Rochester, MN (US)

(21) Appl. No.: 16/749,825

(22) Filed: Jan. 22, 2020

Related U.S. Application Data
(60) Provisional application No. 62/795,540, filed on Jan. 22, 2019.

(51) Int. Cl. G01N 33/543; A61B 5/145; G01N 21/64; B01J 13/02

(52) U.S. Cl. CPC G01N 33/5432; G01N 33/54393; B01J 13/025; A61B 5/14546; G01N 21/6428; G01N 33/54306

(57) ABSTRACT

This document relates to materials, such as microcapsules, and methods for detecting and/or quantifying analytes in a sample such as a biological sample. Microcapsules can comprise a hydrogel shell and an aqueous core comprising one or more analyte capture beads. The aqueous core can further comprise one or more analyte detection beads.

### CROSS-REFERENCE TO RELATED APPLICATIONS

This application claims the benefit of U.S. Provisional Application Ser. No. 62/795,540, filed Jan. 22, 2019. The disclosure of the prior applications is considered part of (and is incorporated by reference in) the disclosure of this application.

### TECHNICAL FIELD

This disclosure generally relates to the detection or quantification of one or more analytes in a biological sample. More particularly, it relates to the detection or quantification of one or analytes in a biological sample using microcapsules.

### BACKGROUND

Detection or quantification of an analyte in a biological sample are commonly performed assays in the biochemical and medical arts. It would be beneficial to have simplified assays for the detection or quantification of an analyte.

### SUMMARY

Various analytes can be present in biological samples, such as blood, urine, spinal fluid, fecal matter, skin scraping, biopsies, and the like, and can be useful as indicators of disease, such as infection, malignancies, autoimmune or allergic reactions or disorders, metabolic diseases, and the like, and/or as monitors of biological processes. For example, cytokines are one such analyte produced by leukocytes in blood, and can be used as indicators of malignancies or infections. Disclosed herein are methods for detecting the presence or absence of, and/or quantifying, such as by immunosensing, analytes, such as, e.g., cytokines, in biological samples. In some instances the biological samples can be unprocessed or minimally processed, such as whole, unprocessed human blood. In some instances, microfluidic droplet generation can be employed to fabricate microcapsules (e.g., ˜400 μm diameter) with a hydrogel shell and an aqueous core containing sensing microbeads and/or nanobeads and optionally, detecting microbeads and/or nanobeads. The hydrogel shell can be poly(ethylene glycol) forming a thin (˜10 μm) immunoisolation layer which can protect antibody-modified microbeads or nanobeads and optionally antibody-modified microbeads and/or nanobeads inside the microcapsule from fouling, such as by immune cells present in the biological sample, such as an unprocessed or minimally processed sample, on the outside of the microcapsule. The microbeads and/or nanobeads can in some instances be functionalized with antibodies against cytokines of interest, such as, e.g., interferon (IFN)-γ and tumor necrosis factor (TNF)-α. While nonfouling, a hydrogel shell can be permeable to analyte molecules, such as cytokine molecules; such molecules can be captured on microbeads and/or nanobeads and detected with fluorescently-labeled secondary antibodies or by using detection microbeads and/or nanobeads. Calibration of encapsulated immunoassays with known concentrations of analytes can be used to quantify target analytes, e.g., IFN-γ (e.g., with a limit of detection of 14.8 μM) and TNF-γ (e.g., with a limit of detection of 14.4 μM). Also disclosed herein is the concept of multi-cytokine detection by fabricating distinct populations of capsules carrying, each carrying a detection moiety for a different analyte of interest, and dispensing the multiple distinct populations of microcapsules into a sample containing none, one, some, or all of the analytes of interest. For example, such a method could include fabricating distinct populations of capsules carrying either anti-IFN-γ or anti-TNF-α microbeads and/or nanobeads and dispensing these capsules into a solution containing neither, one, or both cytokine types. In some embodiments, when placed into whole blood for a period of time (e.g., 16 hours), microcapsules (e.g., microcapsule cores) can be free of leukocytes, effectively protecting sensing beads from the blood components. Further disclosed herein are encapsulated microbeads and/or nanobeads that can be used for detection of IFN-α in blood of patients with latent tuberculosis infection (LTBI) and unexposed healthy controls. When compared to gold standard technology (interferon gamma release assay or IGRA), an immunoassay as disclosed herein can, in some embodiments, accurately predict LTBI diagnosis (e.g., in 11 out of 14 patients). The materials and methods disclosed herein can be a strategy for keeping sensing elements operational in highly fouling complex environments such as blood.

In one aspect, provided herein is a microcapsule having an aqueous core including an analyte capture bead and an analyte detection bead and a hydrogel shell.

In another aspect, provided herein is a composition including a microcapsule.

Implementations can have one or more of the following features. The analyte capture bead can be a microbead and/or nanobead including an analyte-specific binding moiety. The analyte-specific binding moiety can be analyte-specific antibody. The analyte detection bead can be a nanobead including an analyte-specific binding moiety and a detection moiety. The detection moiety can be a fluorophore. The detection moiety can be a fluorescent dye. The analyte detection bead can be a fluorescent nanobead conjugated to an analyte-specific antibody. The aqueous core can include a polymer. The aqueous core can include a densifier. The hydrogel shell can include a cross-linked PEG hydrogel. The hydrogel shell can include a cell-specific capture moiety. The cell-specific capture moiety can include an antibody to a cell surface molecule. The hydrogel shell can include one or more magnetic nanoparticles.

Also provided herein is a method of making a microcapsule including mixing, to form a core-shell mixture, a core solution comprising one or more analyte capture beads and one or more analyte detection beads, and a shell solution comprising a hydrogel precursor, pushing the core-shell mixture through an orifice into a first organic phase to form droplets of the core-shell mixture, passing the droplets into a second organic phase comprising a cross-linker, cross-linking the hydrogel precursor to form microcapsules having hydrogel shells, and collecting the microcapsules.

Implementations can have one or more of the following features. The method can be performed in a microfluidic device. The first organic phase can include a first carrier oil and a surfactant. The first carrier oil in the first organic phase can be selected from the group consisting of mineral oil, a fluorinated oil, and mixtures thereof. The first carrier oil in the first organic phase can be mineral oil. The surfactant in the first organic phase can be selected from the group consisting of sorbitan monooleate, polysorbate 20, and 4-(1,1,3,3-Tetramethylbutyl)phenyl-polyethylene glycol, t-Octylphenoxypolyethoxyethanol, Polyethylene glycol tert-octylphenyl ether, and mixtures thereof. The surfactant in the first organic phase can be sorbitan monooleate. The second organic phase can include a second carrier oil and a surfactant. The second carrier oil in the second organic phase can be selected from the group consisting of mineral oil, a fluorinated oil, and mixtures thereof. The second carrier oil in the second organic phase can be mineral oil. The surfactant in the second organic phase can be selected from the group consisting of sorbitan monooleate, polysorbate 20, and 4-(1,1,3,3-Tetramethylbutyl)phenyl-polyethylene glycol, t-Octylphenoxypolyethoxyethanol, Polyethylene glycol tert-octylphenyl ether, and mixtures thereof. The surfactant in the second organic phase can be sorbitan monooleate. The second organic phase can include an emulsion. The emulsion can include water and the cross-linker. The cross-linker can be selected from dithiothreitol, octanedithiol, and mixtures thereof. The cross-linker can be dithiothreitol (DTT).

Also provided here in is a method of detecting the presence or absence of an analyte, comprising obtaining a sample incubating one or more microcapsules, or a composition including microcapsules, in the sample, retrieving the one or more microcapsules from the sample, and imaging the one or more microcapsules to detect the presence or absence of the analyte.

Implementations can have one or more of the following features. The sample can be whole blood. The analyte is a cytokine. The method can further include quantifying an amount of the analyte in the sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DESCRIPTION OF THE DRAWINGS

FIG. 1 is a schematic of a close-up view of exemplary microcapsules immersed in blood.

FIG. 2 is a schematic of an exemplary workflow of detecting an analyte with microcapsules.

FIG. 3 is a schematic of an exemplary microfluidic device for fabrication of microcapsules.

FIG. 4 is a 3D rendering of a microfluidic device fabricating microcapsules.

FIG. 5A is an image of exemplary microcapsules in whole blood. The dashed yellow line indicates the shell. The scale bar is 200 μm.

FIG. 5B is an image of exemplary microcapsules recovered from simulated blood. The scale bar is 200 μm.

FIG. 5C is a fluorescence image of an exemplary microcapsule sensing INF-γ (green punctates). The shell is seen as a red outline. The scale bar is 200 μm.

FIG. 6A is an image of dual cytokine detection using a mixed population of exemplary microcapsules containing IFN-γ (green punctates) and TNF-α (red punctates) specific microbeads. The scale bar is 200 μm.

FIG. 6B is a close-up image of an exemplary microcapsule sensing IFN-γ. The scale bar is 200 μm.

FIG. 6C is a close-up image of an exemplary microcapsule sensing TFN-α. The scale bar is 200 μm.

FIG. 7A is an IFN-γ calibration curve.

FIG. 7B is a TNF-α calibration curve.

FIG. 8 is a graph showing mean fluorescence intensity.

FIG. 9A is a pie chart showing the results of IGRA assays.

FIG. 9B is a pie chart showing the results of assays with exemplary microcapsules.

FIG. 10 shows representative signals from microcapsules incubated with QFT (+) samples.

FIG. 11 shows representative signals from microcapsules incubated with QFT (−) samples.

FIG. 12 is a fluorescence microscopy image illustrating absence (left) and presence (right) of a target analyte.

FIG. 13 is a flow chart of a microfluidic device fabrication process.

FIG. 14 is a graph plotting the mean fluorescence intensity over time of microcapsules.

FIG. 15A is a graph plotting the mean fluorescence intensity over time of fluorescent particles diffusing into and out of microcapsules.

FIG. 15B is a time-course fluorescence microscopy image illustrating the ability of large molecules to diffuse into and out of microcapsules.

FIG. 16 is a graph plotting the mean fluorescence intensity of a sample based on the number of microcapsules therein.

FIG. 17 is a graph and paired fluorescence microscopy images of the fluorescence intensity of microcapsule populations that were labeled with antibodies pre- and post-crosslinking.

FIG. 18 is fluorescence microscopy of microcapsules challenged with increasing concentrations of the target analyte, a cytokine.

FIG. 19 is a graph plotting the mean fluorescence intensity of microcapsules that were incubated with cytokines that did or did not match the antibodies on the capture bead.

### DETAILED DESCRIPTION

This document is related to the detection of one or more analytes in a biological sample. In some embodiments, provided herein are microcapsules for the detection of one or more analytes in a biological sample.

Since the demonstration of a glucose-selective electrode by Updike and Hicks in 1967 (Updike S J, Hicks G P. 1967. The enzyme electrode. Nature 214:986-8), the development of biosensors has often proceeded along with the development of biological interfaces to ensure and extend biosensor operation in a fouling environment (e.g., whole blood) (Rocchitta G, Spanu A, Babudieri S, Latte G, Madeddu G, Galleri G, Nuvoli S, Bagella P, Demartis M I, Fiore V, Manetti R and Serra P A. 2016. Enzyme Biosensors for Biomedical Applications: Strategies for Safeguarding Analytical Performances in Biological Fluids. Sensors-Basel 16). In some cases, sensing elements (e.g. enzymes) have been protected from the fouling biological environment by entrapment within polyacrylamide gels or by membranes or by polyelectrolyte coatings (Sassolas A, Blum L J, Leca-Bouvier B D. 2012. Immobilization strategies to develop enzymatic biosensors. Biotechnol Adv 30:489-511; Kim J H, Choi D C, Yeon K M, Kim S R, Lee C H. 2011. Enzyme-Immobilized Nanofiltration Membrane To Mitigate Biofouling Based on Quorum Quenching. Environ Sci Technol 45:1601-7; Ambrózy A, Hlavatá, L., & Labuda, J. (n.d.). 2013. Protective membranes at electrochemical biosensors. Acta Chimica Slovaca, 6:35-41). Poly (ethylene glycol) (PEG) hydrogels have been utilized for minimizing fouling of biosensors (Matharu Z, Enomoto J, Revzin A. 2013. Miniature Enzyme-Based Electrodes for Detection of Hydrogen Peroxide Release from Alcohol-Injured Hepatocytes. Anal Chem 85:932-9; Yan J, Pedrosa V A, Enomoto J, Simonian AL, Revzin A. 2011. Electrochemical biosensors for on-chip detection of oxidative stress from immune cells. Biomicrofluidics 5; Koh W G, Pishko M. 2005. Immobilization of multi-enzyme microreactors inside microfluidic devices. Sensor Actuat B-Chem 106:335-42; Yan J, Pedrosa V A, Simonian A L, Revzin A. 2010. Immobilizing Enzymes onto Electrode Arrays by Hydrogel Photolithography to Fabricate Multi-Analyte Electrochemical Biosensors. Acs Appl Mater Inter 2:748-55). For example, PEG hydrogel coatings have been used to improve the lifetime and operation of glucose-sensing electrodes developed by Heller and colleagues (Ohara T J, Rajagopalan R, Heller A. 1994. Wired Enzyme Electrodes for Amperometric Determination of Glucose or Lactate in the Presence of Interfering Substances. Anal Chem 66:2451-7). PEG hydrogels have also been fabricated into a variety of objects by photolithography or stop-flow lithography and have been loaded with biorecognition elements for biosensing applications (Shin D S, Liu Y, Gao Y D, Kwa T, Matharu Z, Revzin A. 2013. Micropatterned Surfaces Functionalized with Electroactive Peptides for Detecting Protease Release from Cells (vol 85, pg 220, 2013). Anal Chem 85:3795; Le Goff G C, Srinivas R L, Hill W A, Doyle P S. 2015. Hydrogel microparticles for biosensing. Eur Polym J 72:386-412).

Droplet microfluidics represents another microfabrication approach that has been used for high-throughput cell encapsulation and biochemical assays (Huang H S, Yu Y, Hu Y, He X M, Usta O B, Yarmush M L. 2017. Generation and manipulation of hydrogel microcapsules by droplet-based microfluidics for mammalian cell culture. Lab Chip 17:1913-32; Shang L R, Cheng Y, Zhao Y J. 2017. Emerging Droplet Microfluidics. Chem Rev 117:7964-8040). Typically, droplet microfluidics devices produce water-in-oil emulsions, but this fabrication strategy can be adapted to produce hydrogel capsules (Guillot P, Colin A. 2005. Stability of parallel flows in a microchannel after a T junction. Phys Rev E 72; Siltanen C, Yaghoobi M, Haque A, You J, Lowen J, Soleimani M and Revzin A. 2016. Microfluidic fabrication of bioactive microgels for rapid formation and enhanced differentiation of stem cell spheroids. Acta Biomater 34:125-32). Potential advantages of microfluidic fabrication can include monodispersity of capsules size and also complexity of capsule composition (Headen D M, Aubry G, Lu H, Garcia A J. 2014. Microfluidic-Based Generation of Size-Controlled, Biofunctionalized Synthetic Polymer Microgels for Cell Encapsulation. Adv Mater 26:3003-8). For example, there have been reports of core-shell microcapsules produced using droplet microfluidics as miniaturized bioreactors for cultivation of yeast and mammalian cells (Ekanem E E, Zhang Z, Vladisavljevic G T. 2017. Facile microfluidic production of composite polymer core-shell microcapsules and crescent-shaped microparticles. J Colloid Interface Sci 498:387-94; Agarwal P, Zhao S T, Bielecki P, Rao W, Choi J K, Zhao Y, Yu J and He X. 2013. One-step microfluidic generation of pre-hatching embryo-like core-shell microcapsules for miniaturized 3D culture of pluripotent stem cells. Lab Chip 13:4525-33; Kim C, Chung S, Kim Y E, Lee K S, Lee S H, Oh K W and Kang J Y. 2011. Generation of core-shell microcapsules with three-dimensional focusing device for efficient formation of cell spheroid. Lab Chip 11:246-52). Fabrication of microparticles with an aqueous core and hydrogel shell for entrapment of cells and formation of spheroids has been reported (Siltanen C, Diakatou M, Lowen J, Haque A, Rahimian A, Stybayeva G and Revzin A. 2017. One step fabrication of hydrogel microcapsules with hollow core for assembly and cultivation of hepatocyte spheroids. Acta Biomater 50:428-36). Described herein is the use of core-shell microcapsules for entrapment of antibody-modified microbeads and/or nanobeads and detection microbeads and/or nanobeads for detection of analytes, such as cytokines, in blood.

Various analytes of interest are present in biological samples, such as in urine, blood, spinal fluid, and the like. For example, in blood, cytokines are released by immune cells responding to infections, malignancies, or autoimmune disorders (Chung K F. 2009. Cytokines. Asthma and Copd: Basic Mechanisms and Clinical Management, 2nd Edition:327-41). Interferon (IFN)-γ and TNF-α are some of the most common cytokines released by leukocytes. The former is of T-cell origin and may be indicative of T-cell response to antigenic stimulation, while the latter indicates a general inflammatory response and can be produced by a number of leukocyte subtypes, including T-cells and monocytes (Cavalcanti Y V, Brelaz M C, Neves J K, Ferraz J C, Pereira V R. 2012. Role of TNF-Alpha, IFN-Gamma, and IL-10 in the Development of Pulmonary Tuberculosis. Pulm Med 2012:745483; Sasiain M C, de la Barrera S, Fink S, Finiasz M, Aleman M, Farina M H, Pizzariello G and Valdez R. 1998. Interferon-gamma (IFN-gamma; IFN-γ) and tumor necrosis factor-alpha (TNF-alpha; TNF-α) are necessary in the early stages of induction of CD4 and CD8 cytotoxic T cells by Mycobacterium leprae heat shock protein (hsp) 65 kD. Clin Exp Immunol 114:196-203). IFN-γ is particularly important as a diagnostic marker of latent tuberculosis infection (LTBI), which is detected by either an ELISpot- or an ELISA-based interferon gamma release assay (IGRA) (Chee C B, KhinMar K W, Gan S H, Barkham T M, Pushparani M, Wang Y T. 2007. Latent tuberculosis infection treatment and T-cell responses to Mycobacterium tuberculosis-specific antigens. Am J Respir Crit Care Med 175:282-7; Leem A Y, Song J H, Lee E H, Lee H, Sim B, Kim S Y, Chung K S, Kim E Y, Jung J Y, Park M S, Kim Y S, Chang J and Kang Y A. 2018. Changes in cytokine responses to TB antigens ESAT-6, CFP-10 and TB 7.7 and inflammatory markers in peripheral blood during therapy. Sci Rep 8:1159; Pai M, Denkinger C M, Kik S V, Rangaka M X, Zwerling A, Oxlade O, Metcalfe J Z, Cattamanchi A, Dowdy D W, Dheda K, Banaei N. 2014. Gamma interferon release assays for detection of Mycobacterium tuberculosis infection. Clin Microbiol Rev 27:3-20). However, both IGRAs are multi-step assays that involve removal of blood cells followed by a complex multi-step ELISpot (enzyme-linked immune absorbent spot) or ELISA (enzyme-linked immunosorbent assay) detection method (Lalvani A. 2007. Diagnosing tuberculosis infection in the 21st century: new tools to tackle an old enemy. Chest 131:1898-906).

In one aspect, provided herein are microcapsules carrying microbeads and/or nanobeads for the detection and/or quantification of one or more analytes in a biological sample, such as minimally processed blood. In some aspects, microcapsules described herein comprise an aqueous core and a hydrogel shell. In some aspects, the aqueous core can comprise an analyte capture bead and an analyte detection bead. In some aspects, the aqueous core can comprise an analyte capture bead and does not comprise an analyte detection bead. In some cases, an analyte capture bead and/or an analyte detection bead can be a microbead and/or a nanobead. In some aspects, microcapsules provided herein can include antibody(Ab)-modified microbeads and/or nanobeads for detection of IFN-γ and TNF-α in minimally processed blood.

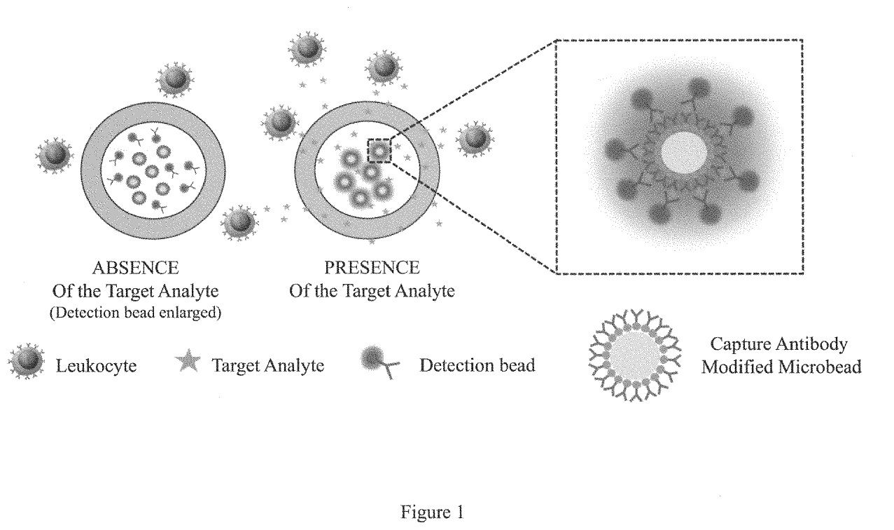

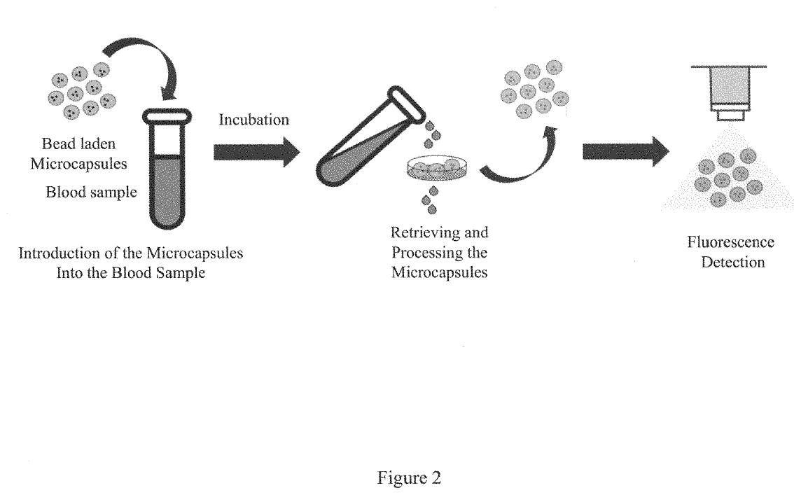

In some aspects, isolating analyte capture microbeads and/or nanobeads and analyte detection microbeads and/or nanobeads inside core-shell microcapsules eliminates the need for multiple sample preparation steps and allows target analytes (e.g., cytokines) to be sampled in unprocessed blood (see FIGS. 1 and 2). FIG. 1 shows a close-up diagram of exemplary microcapsules immersed in blood. Target analytes (e.g., cytokines from stimulated leukocytes) can diffuse into the core of a microcapsule and can be captured by antibody-modified beads residing in the core. Presence of the target analyte can be revealed by capture of the target analyte by one or more analyte detection beads. The fluorescence intensity of encapsulated microbeads and/or nanobeads can, in some embodiments, be correlated with concentration of the target analyte in blood. FIG. 2 illustrates one exemplary workflow of detecting a target analyte with sensing microcapsules. The microcapsules can be added into a biological sample (e.g., a heparinized tube containing patient's blood) and can be incubated overnight under cell culture conditions. Upon completion of the incubation, the microcapsules can be separated from the biological sample (e.g., using a strainer with 40-200 μm mesh size). Microcapsules can then be imaged to quantify fluorescence intensity. In some aspects, analyte capture microbeads and/or nanobeads can be isolated inside core-shell microcapsules without analyte detection microbeads and/or nanobeads inside core-shell microcapsules. In some such cases, an additional step of added the analyte detection microbeads and/or nanobeads to the sample can be utilized, allowing the detection microbeads and/or nanobeads to diffuse across the hydrogel shell and into the core of the microcapsule to bind and detect an analyte bound to the capture microbeads and/or nanobeads. In some such cases, an additional step of adding a fluorescently labeled detection antibody to the microcapsules (e.g., microcapsules separated from the biological sample) can be utilized, allowing the detection antibody to diffuse across the hydrogel shell and into the core of the microcapsule to bind and detect an analyte bound to the capture microbeads and/or nanobeads. However, such methods still eliminate the need for other sample preparation steps.

An analyte detected by microcapsules as described herein can be any analyte of interest that can be detected an analyte-specific binding moiety (e.g., an antibody). For example, an analyte can be a cytokine, or a hormone, or an eicosanoid, or the like, or any combinations thereof. In some embodiments, an analyte can be selected from a cytokine, a hormone, or an eicosanoid, or the like, or any combinations thereof. Non-limiting examples of hormones include a peptide or protein hormone (e.g., insulin, glucagon, adrenocorticotropic hormone, oxytocin, vasopressin, parathyroid hormone, atrial-natriuretic peptide, leptin, growth hormone), and a steroid hormone (e.g., a glucocorticoid, a mineralocorticoid, an androgen, an estrogen, a progesterone, a vitamin D). Non-limiting examples of eicosanoids include a prostaglandin, a thromboxane, a leukotriene, a lipoxin, a resolvin, and an eoxin. As another example, an analyte can be nucleic acid (e.g., DNA (e.g., circulating DNA) or RNA (e.g., microRNA, mRNA)).

In some embodiments, an analyte of interest can be a cytokine. Cytokines are proteins that are involved in cell signaling. Cytokines can be involved in, for example, autocrine signaling, paracrine signaling, and endocrine signaling. Cytokines are generally small proteins, from about 5-20 kDa in size. Non-limiting examples of cytokines include a lymphokine, an interferon, an interleukin, a tumor necrosis factor, or a chemokine. In some cases, cytokines can be classified by function, such as cytokines that: enhance cellular immune responses (e.g., TNF-α, IFN-γ) or enhance antibody responses (e.g., TGF-β, IL-4, IL-10, IL-13). In some embodiments, an analyte can be IFN-γ. In some embodiments, an analyte can be TFN-α.

An analyte-specific binding moiety as described herein can be any analyte-specific binding moiety that binds to an analyte of interest. For example, an analyte-specific binding moiety can be an antibody, an antibody fragment, or an aptamer (e.g., a nucleic acid or a peptide aptamer). In some embodiments, an analyte-specific binding moiety can be a nucleic acid (e.g., a nucleic acid that has a region complementary to a region in an analyte nucleic acid). It will be appreciated that an analyte-specific binding moiety can be generated by any appropriate method. In some cases, an analyte-specific binding moiety on an analyte capture bead is the same as an analyte-specific binding moiety on an analyte detection bead. In some cases, an analyte-specific binding moiety on an analyte capture bead is different from an analyte-specific binding moiety on an analyte detection bead (e.g., antibodies that bind to different epitopes on an analyte).

In some embodiments, an analyte-specific binding moiety can be an antibody. Antibodies are well-known in the medical arts to be useful in the detection of various analytes of interest, including proteins, Antibodies can bind very specifically to any analyte of interest. In some embodiments, an antibody can be monoclonal. In some embodiments, an antibody can be polyclonal. In some embodiments, an antibody to an analyte of interest can be purchased. In some embodiments, an antibody to an analyte of interest can be generated by any method known in the art. In some embodiments, an analyte-specific binding moiety can be an anti-IFN-γ antibody. In some embodiments, an anti-IFN-γ antibody can be any antibody that binds to the human IFN-γ protein sequence, such as the sequence of GenBank accession number CAA31639. Non-limiting examples of anti-IFN-γ antibodies include AbCam product Nos. ab218426, ab9657, ab25101, ab9801, ab9805, ab7740, ab9918, ab8096, ab180547, ab185751, ab7372, ab11855, ab83136, ab185788, ab7361, ab9658, ab24968, ab136387, and ab136390; BioLegend catalog Nos. 714006, 575302, 575309, 598802, 502529, 502503, 502505, 502521, 51790, 506510, 50780, 507501, and 502401; R&D Systems catalog Nos. FAB6731S-100UG, FAB6731N-100UG, FAB6731R-100UG, FAB773G-100UG, IC285R-100UG, IC285U-100UG, and MAB285-SP; and ThermoFisher Scientific catalog Nos. MHCIFG04-3, 11-7311-82, 11-7319-82, M700A, M701, 25-7311-82, 48-7311-82, 45-7311-82, 25-7319-82, 45-7319-42, 47-7319-42, MA5-23718, M701B, and MHCIFG29. Additional non-limiting examples of anti-IFN-γ antibodies can be found elsewhere, for example, in the product catalogs of Abcam, Cell Signaling Technology, Sigma Aldrich (Millipore Sigma), EMD Millipore (Millipore Sigma), BD Biosciences, Thermo Fisher Scientific, New England Biolabs, or any other antibody vendor. In some embodiments, an analyte-specific binding moiety can be an anti-TNF-α antibody. In some embodiments, an anti-TNF-α antibody can be any antibody that binds to the human TNF-α protein sequence, such as the sequence of Uniprot accession number P01375. Non-limiting examples of anti-TNF-α antibodies include AbCam product Nos. ab220210, ab6671, ab1793, ab8348, ab9739, ab66579, ab199013, ab34674, ab9635, ab9579, ab183218, ab215188, ab9755, ab11564, ab225576, ab34719, ab212899, ab183896, ab9809, and ab7742; BioLegend catalog Nos. 901201, 510801, 502913, 502903, 502906, 502929, 502901, 506307, and 506343; R&D Systems catalog Nos. AF-410-SP, MAB610-SP, AF-210-SP, BAF210, BAF410, AF-510-SP, MAB210-SP, MAB4101-SP MAB510-SP, 510-RT-010, 510-RT-010/CF, MAB2101-SP, IC210F, AB-210-NA; and ThermoFisher Scientific catalog Nos. P300A, MM350C, 11-7349-82, 11-7321-82, 56-7349-42, 25-7349-82, 48-7349-42, 12-7321-82, 45-7349-42, 17-7349-41, MA5-23720, AHC3912, 710288, M2TNFAI, M303, M302, MTNFAI, AHC3419. Additional non-limiting examples of anti-TNF-a antibodies can be found elsewhere, for example, in the product catalogs of Abcam, Cell Signaling Technology, Sigma Aldrich (Millipore Sigma), EMD Millipore (Millipore Sigma), BD Biosciences, Thermo Fisher Scientific, New England Biolabs, or any other antibody vendor. In some embodiments, a commercially available antibody can be purchased including conjugation to another molecule, such as biotin or a fluorescent dye. In some embodiments, a commercially available antibody can be purchased unconjugated.

An analyte capture bead can be any appropriate bead to which an analyte-specific binding moiety is conjugated. In some embodiments, an analyte capture bead can have a diameter of about 3 μm to about 7 μm (e.g., about 3 μm to about 5 μm, about 4 μm to about 6 μm, or about 5 μm to about 7 μm). An analyte capture bead can include a bead core and an analyte-specific capture moiety. An analyte capture bead can include a bead core, a linker, and an analyte-specific binding moiety. In some embodiments, a bead core can be any appropriate bead core. In some embodiments, a bead core is not fluorescent. In some embodiments, a bead core can include a fluorophore. In some embodiments, a fluorophore can be part of the bead core. In some embodiments, a bead core can be coated with a fluorophore. In some embodiments, a fluorophore can be attached to a bead core via a linker. In some embodiments, an analyte capture bead is not fluorescent. In some embodiments, an analyte capture bead does not include a fluorescent dye. In some embodiments, a bead core is a microparticle or a nanoparticle. In some embodiments, a bead core comprises polystyrene. In some embodiments, a bead core is a magnetic bead (e.g., made of iron oxide particles). In some embodiments, a bead core comprises latex. In some embodiments, a bead core can be coated with a linker. A linker can be any appropriate linker that allows the attachment of an analyte-specific binding moiety. For exam5ple, a bead core can be coated with avidin. For example, a bead core can be coated with streptavidin. As another example, a bead core can be coated with biotin. In some cases, a bead core can be purchased from a vendor already coated with a linker. In some cases, a bead core can be coated with a linker using any appropriate method. In some aspects, an analyte capture bead can bind an analyte inside the microcapsule. In some aspects, an analyte capture bead can immobilize an analyte inside the microcapsule.

An analyte detection bead can be any appropriate bead to which an analyte-specific binding moiety is conjugated. In some embodiments, an analyte capture bead can have a diameter of about 0.1 μm to about 0.7 μm (e.g., about 0.1 μm to about 0.3 μm, about 0.1 μm to about 0.5 μm, about 0.2 μm to about 0.4 μm, about 0.3 μm to about 0.5 μm, about 0.4 μm to about 0.6 μm, or about 0.5 μm to about 0.7 μm). In some embodiments, a bead core is a microparticle or a nanoparticle. An analyte detection bead can include a bead core and an analyte-specific binding moiety. An analyte detection bead can include a bead core, a linker, and an analyte-specific binding moiety. In some embodiments, a bead core can be any appropriate bead core. In some embodiments, a bead core is not fluorescent. In some embodiments, a bead core can include a fluorophore. In some embodiments, a fluorophore can be part of the bead core. In some embodiments, a bead core can be coated with a fluorophore. In some embodiments, a fluorophore can be attached to a bead core via a linker. A fluorophore can be any appropriate fluorophore. In some embodiments, a fluorophore can be Nile Red. In some embodiments, a bead core is a nanoparticle. In some embodiments, a bead core comprises polystyrene. In some embodiments, a bead core is a magnetic bead (e.g., made of iron oxide particles). In some embodiments, a bead core comprises latex. In some embodiments, a bead core can be coated with a linker. A linker can be any appropriate linker that allows the attachment of an analyte-specific binding moiety. For example, a bead core can be coated with avidin. For example, a bead core can be coated with streptavidin. As another example, a bead core can be coated with biotin. In some cases, a bead core can be purchased from a vendor already coated with a linker. In some cases, a bead core can be coated with a linker using any method known in the art.

An analyte-specific binding moiety can be conjugated to a bead (e.g., a capture bead or a detection bead) using any appropriate method. For example, in some embodiments, a streptavidin-coated microbead and/or nanobead can be the bead. Biotinylated anti-IFN-γ or anti-TNF-α Abs can be immobilized on microbeads (e.g., 5-5.9 μm diameter) and/or nanobeads according any appropriate protocol (e.g., those described in Son K J, Rahimian A, Shin D S, Siltanen C, Patel T, Revzin A. 2016. Microfluidic compartments with sensing microbeads and/or nanobeads for dynamic monitoring of cytokine and exosome release from single cells. Analyst 141:679-88; or Son K J, Gheibi P, Stybayeva G, Rahimian A, Revzin A. 2017. Detecting cell-secreted growth factors in microfluidic devices using bead-based biosensors. Microsyst Nanoeng 3). Briefly, prior to conjugation, microbeads and/or nanobeads can be incubated with protein-free blocking buffer (e.g., for 2 h at 4° C.) then washed (e.g., with PBS) (e.g., via a centrifugation at 13,000 rcf for 5 min). In some embodiments, washed streptavidin-microbeads and/or nanobeads can be incubated with biotinylated capture Abs (e.g., in 250 μL 1% BSA in PBS overnight at 4° C.). After Ab immobilization, microbeads and/or nanobeads can be washed (e.g., in PBS by centrifugation) before reconstituting in 2× concentrated core solution.

Non-limiting examples of fluorophores include an Alexa Fluor dye (e.g., Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750), Pacific Blue, coumarin, a BODIPY dye, Pacific Green, Oregon Green, fluorescein, a fluorescein derivative (e.g., FITC), a cyanine dye (e.g., Cy3, Cy5), Pacific Orange, Nile Red, a rhodamine dye (e.g., tetramethylrhodamine), and Texas Red.

In some embodiments, an analyte capture bead and an analyte detection bead can produce a proximity-dependent signal. For example, in some embodiments, an analyte capture bead can include a FRET (fluorescence resonance energy transfer) donor fluorophore, and an analyte detection bead can include a FRET acceptor fluorophore. In some embodiments, an analyte capture bead can include a FRET acceptor fluorophore, and an analyte detection bead can include a FRET donor fluorophore. A FRET pair (e.g., a donor fluorophore and an acceptor fluorophore) can be an acceptable FRET pair. Similarly, in some embodiments, an ALPHA (Amplified Luminescent Proximity Homogenous Assay) bead pair can be used. An ALPHA bead pair can be any appropriate ALPHA bead pair. For example, in some embodiments, an analyte capture bead can be an ALPHA donor bead, and an analyte detection bead can be an ALPHA acceptor bead. In some embodiments, an analyte capture bead can be an ALPHA acceptor bead, and an analyte detection bead can be an ALPHA donor bead. In some embodiments, an ALPHA donor bead can include a photosensitizer that can excite ambient oxygen into a singlet state following high energy irradiation (e.g., at 680 nm). In some embodiments, an ALPHA acceptor bead can include a dye that can be excited by singlet oxygen (e.g., thioxene).

In some aspects, a microcapsule as described herein can include an aqueous core including an analyte capture bead and an analyte detection bead, and a hydrogel shell. In some aspects, a microcapsule as described herein can include an aqueous core including an analyte capture bead and a hydrogel shell, without an analyte detection bead inside the core.

In some embodiments, an aqueous core can be made of core solution. In some embodiments, a core solution can include a polymer. A polymer can be any appropriate polymer. In some embodiments, a polymer can be a PEG (polyethylene glycol). A PEG can be a PEG of any appropriate average molecular weight. In some embodiments, a PEG can be a PEG of an average weight of between about 400 daltons to about 100,000 daltons. For example, a PEG can be a 35 k PEG. In some embodiments a PEG can be a PEG 400, a PEG 600, a 1 k PEG, a PEG 1500, a 3 k PEG, a PEG 3350, a 5 k PEG, a 8 k PEG, a 10 k PEG, a 15 k PEG, a 20 k PEG, a 35 k PEG, a 40 k PEG, a 50 k PEG, a 60 k PEG, a 70 k PEG, a 80 k PEG, a 90 k PEG, or a 100 k PEG. A PEG can be present in a core solution in any appropriate concentration. In some embodiments, a PEG can be present in a core solution in a concentration of about 4% to about 20% (w/v) (e.g., about 4%, to about 18%, about 4% to about 16%, about 4% to about 14%, about 4% to about 12%, about 4% to about 10%, about 4% to about 8%, about 4% to about 6%, about 6% to about 20%, about 6% to about 18%, about 6% to about 16%, about 6% to about 14%, about 6% to about 12%, about 6% to about 10%, about 6% to about 8%, about 8% to about 20%, about 8% to about 18%, about 8% to about 16%, about 8% to about 14%, about 8% to about 12%, about 8% to about 10%, about 10% to about 20%, about 10% to about 18%, about 10% to about 16%, about 10% to about 14%, about 10% to about 12%, about 12% to about 20%, about 12% to about 18%, about 12% to about 16%, about 12% to about 14%, about 14% to about 20%, about 14% to about 18%, about 14% to about 16%, about 16% to about 20%, about 16% to about 18%, about 18% to about 20%, about 4%, about 5%, about 6%, about 7%, about 8%, about 9%, about 10%, about 11%, about 12%, about 13%, about 14%, about 15%, about 16%, about 17%, about 18%, about 19%, or about 20% (w/v)). In some embodiments, a polymer can diffuse out of a microparticle, in whole or in part, following fabrication. In some embodiments, a core solution can include a densifier. Without being bound by any particular theory, it is believed that a densifier can help to prevent precipitation of the beads. A densifier can be any appropriate densifier. In some embodiments, a densifier can be a solution of iodixanol (e.g., OPTIPREP™). In some embodiments, a densifier can be present in a core solution in a concentration of about 14% to about 60% (v/v) (e.g., about 14% to about 16%, about 14% to about 18%, about 14% to about 20%, about 16% to about 18%, about 18% to about 22%, about 15% to about 60%, about 20% to about 60%, about 30% to about 60%, about 40% to about 60%, about 50% to about 60%, about 15% to about 20%, about 15% to about 30%, about 15% to about 40%, about 15% to about 50%, about 20% to about 30%, about 20% to about 40%, about 20% to about 50%, about 30% about 40%, about 30% about 50%, about 40% to about 50%, about 14%, about 15%, about 16%, about 17%, about 18%, about 19%, about 20%, about 21%, about 22%, about 25%, about 30%, about 35%, about 40%, about 45%, about 50%, about 55%, or about 60% (v/v)). In some embodiments, core solution can include a cell culture media. A cell culture media can be any appropriate cell culture media. In some embodiments, cell culture media can be phosphate-buffered saline (PBS). In some embodiments, cell culture media can be 1% bovine serum albumin (BSA) in PBS. In some embodiments, core solution can include a plurality of analyte capture beads. In some embodiments, an aqueous core can include a plurality of analyte detection beads. In some embodiments, core solution can include a plurality of analyte capture beads and a plurality of analyte detection beads. In some embodiments, about 70 to about 240 (e.g., about 70 to about 80, about 70 to about 90, about 70 to about 100, about 70 to about 120, about 70 to about 140, about 70 to about 180, about 70 to about 200, about 70 to about 220, about 70 to about 240, about 80 to about 90, about 80 to about 100, about 80 to about 120, about 80 to about 140, about 80 to about 180, about 80 to about 200, about 20 to about 220, about 80 to about 240, about 100 to about 120, about 100 to about 140, about 100 to about 160, about 100 to about 180, about 100 to about 200, about 100 to about 220, about 100 to about 240, about 120 to about 140, about 120 to about 160, about 120 to about 180, about 120 to about 200, about 120 to about 220, about 120 to about 240, about 140 to about 160, about 140 to about 180, about 140 to about 200, about 140 to about 220, about 140 to about 240, about 160 to about 180, about 160 to about 200, about 160 to about 220, about 160 to about 240, about 180 to about 200, about 180 to about 220, about 180 to about 240, about 200 to about 220, about 200 to about 240, about 220 to about 240, about 70, about 80, about 90, about 100, about 120, about 140, about 160, about 180, about 200, about 220, or about 240) analyte detection beads can be present for every analyte capture bead.

A hydrogel shell can include any appropriate components. In some embodiments, a hydrogel shell can include crosslinked polymers. In some embodiments, a hydrogel shell can include a crosslinked polymer selected from a polyurethane, an acrylate, a polyethyleneglycol, a polydimethylsiloxane, and a polyisobutylene. In some embodiments, a hydrogel shell can include crosslinked PEG. In some embodiments, a hydrogel shell can include a linker. A linker can be any appropriate linker that allows for the attachment of a cell-specific binding moiety. In some embodiments, a linker can include avidin. In some embodiments, a linker can include streptavidin. In some embodiments, a hydrogel shell can include a cell-specific binding moiety. A cell-specific binding moiety can be any appropriate cell-specific binding moiety. In some examples, a cell-specific binding moiety can be an antibody, an antibody fragment, or an aptamer. In some embodiments, a cell-specific binding moiety can be an antibody. In some embodiments, a cell-specific binding moiety can be a biotin-labeled antibody. In some embodiments, a cell-specific binding moiety can be an antibody that binds specifically to leukocytes. Without being bound by any theory, it is believed that bringing cells closer to sensor can increase local concentration of secreted signals and can enable more sensitive and specific detection of analytes (e.g., cytokines or other cell-secreted products). In some embodiments, a hydrogel shell can include magnetic nanoparticles. A magnetic nanoparticle can be any appropriate magnetic nanoparticle. In some embodiments, a magnetic nanoparticle can be iron oxide. Without being bound by any theory, it is believed that incorporation of magnetic nanoparticles can be used to retrieve the microcapsules from a biological sample. For example, standard magnets of the type use for magnetic cell sorting will be used to retrieve capsules from the sample. In some embodiments, a microcapsule, can contain from about 1-5 wt % magnetic nanoparticles, such as iron nanoparticles. In some embodiments, a hydrogel shell of a microcapsule, can contain from about 1-5 wt % magnetic nanoparticles, such as iron nanoparticles.

In some embodiments, a hydrogel shell can be made from crosslinking of polymers in a shell solution. In some embodiments, a shell solution can include a hydrogel precursor. A hydrogel precursor can be any appropriate hydrogel precursor polymer that can be crosslinked to form a hydrogel, such as, e.g., a polymer selected from a polyurethane, an acrylate, a polyethyleneglycol, a polydimethylsiloxane, and a polyisobutylene, and the like. In some embodiments, a hydrogel precursor can be a maleimide functionalized polymer. In some embodiments, a hydrogel precursor can be a maleimide functionalized PEG (e.g., a 4-arm maleimide functionalized PEG). A hydrogel precursor can be present in a shell solution in any appropriate amount. In some embodiments, a hydrogel precursor can be present in a shell solution in an amount of about 4-12% (w/v) (e.g., about 4% to about 6%, about 4% to about 8%, about 4% to about 10%, about 6% to about 8%, about 6% to about 10%, about 6% to about 12%, about 8% to about 10%, about 8% to about 12%, about 10% to about 12%, about 4%, about 5%, about 6%, about 7%, about 8%, about 9%, about 10%, about 11%, or about 12% (w/v)). In some embodiments, a shell solution can include a crosslinking facilitator (e.g., for a crosslinking reaction). In some embodiments, a crosslinking facilitator can be triethanolamine (TEA). In some embodiments, a crosslinking facilitator can be present in a shell solution in any appropriate amount. In some embodiments, a crosslinking facilitator can be present in a shell solution an amount of about 10 mM to about 20 mM (e.g., about 10 mM to about 12 mM, about 12 mM to about 14 mM, about 14 mM to about 16 mM, about 16 mM to about 18 mM, about 18 mM to about 20 mM, about 10 mM, about 11 mM, about 12 mM, about 13 mM, about 14 mM, about 15 mM, about 16 mM, about 17 mM, about 18 mM, about 19 mM, or about 20 mM). In some embodiments, a shell solution can include cell culture media. A cell culture media can be any appropriate cell culture media. In some embodiments, cell culture media can be phosphate-buffered saline (PBS). In some embodiments, cell culture media can be 1% bovine serum albumin (BSA) in PBS. In some embodiments, the cell culture media in a shell solution can be the same type of cell culture media as in a core solution.

A biological sample can be any appropriate biological sample. For example, a biological sample can be selected from the group consisting of whole blood, blood components (e.g., serum, plasma, red blood cells, white blood cells, or platelets), urine, stool (e.g., liquefied stool), saliva, cerebrospinal fluid, and amniotic fluid. In some embodiments, a biological sample can be minimally processed, such that the majority of the natural or original sample components remain present or in an unchanged state, and only a small amount of filtration, removal, or alteration of the sample has occurred. One example of a minimally processed sample is whole blood that has been treated with heparin. In some embodiments, a sample can be completely unprocessed. In some embodiments, a biological a sample can be whole blood. In some embodiments, it may be desired that the sample be a liquid sample. Thus, samples that are not typically liquid, such as tissue scrapings or samples, stool, and the like, may be suspended in a liquid (e.g., liquefied) and/or processed for appropriate suspension in a liquid for analysis.

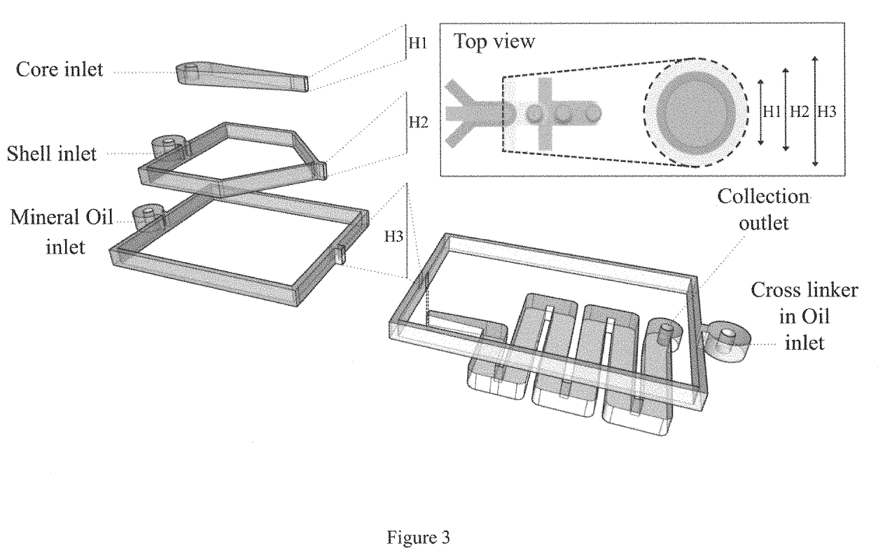

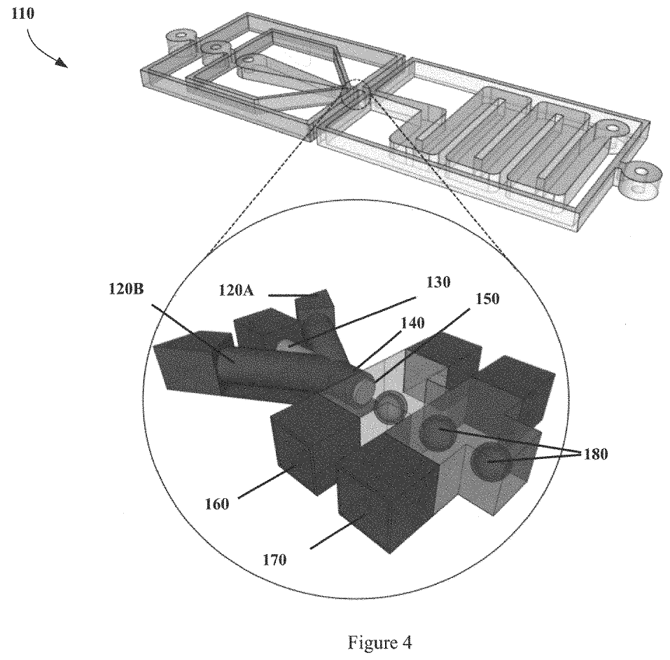

Also provided herein are methods of fabricating any of the microcapsules as described herein. In some embodiments, a method can include mixing a core solution including an analyte capture bead and an analyte detection bead and a shell solution including a hydrogel precursor to form a core-shell mixture, pushing the core-shell mixture through an orifice into a first organic phase to form droplets of the core-shell mixture, passing the droplets into a second organic phase comprising a cross-linker, cross-linking the shell to form microcapsules, and collecting the microcapsules. In some embodiments a core-shell mixture can be a heterogeneous mixture. In some embodiments, a core-shell mixture can include a central portion of core solution surrounded by a sheath (if a stream) or a shell (if a droplet) of shell solution. It will be understood that some mixing could occur at a solution-solution interface. In some embodiments, a core solution can be any of the core solutions as described herein. In some embodiments, an analyte capture bead can be any of the analyte capture beads as described herein. In some embodiments, an analyte detection bead can be any of the analyte detection beads as described herein. In some embodiments, a shell solution can be any of the shell solutions as described herein. In some embodiments, a method of fabricating any of the microcapsules as described herein can be performed in a microfluidic device. FIGS. 3 and 4 illustrate exemplary microfluidic devices. FIG. 3 illustrates an exemplary microfluidic device. A droplet generator can include 4 inlet channels and a serpentine that leads to droplet collection port. The core, shell, and oil channels can have heights of 120 μm (H1), 200 μm (H2), and 300 μm (H3), respectively. The top view panel describes how channels of different dimensions can correlate to the core and shell components of microcapsules. FIG. 4 is a 3D rendering to help visualize the process of fabricating core-shell microcapsules. A core flow stream can contain beads, high molecular weight PEG and a densifier. The shell stream can include cross-linkable 4Arm PEG-Maleimide (Mal). Upon ejection from the nozzle into the oil phase, the liquid in the shell can wrap around the material in the core. Subsequently, PEG-4Mal in the shell can form a gel after interaction with oil/cross-linker stream. Large molecular weight PEG molecules lacking functional groups can diffuse out of the core and can be replaced by aqueous environment within an hour of capsule fabrication.

In some embodiments, a first organic phase can include a first carrier oil. In some embodiments, a first organic phase can include a surfactant. In some embodiments, a second organic phase can include a second carrier oil. In some embodiments, a second organic phase can include a surfactant. A carrier oil (e.g., first carrier oil, second carrier oil) can be any appropriate carrier oil. In some embodiments, a carrier oil can be mineral oil. In some embodiments, a carrier oil can be a fluorinated oil. In some embodiments, the carrier oil in a first organic phase can be the same type of oil as the carrier oil in a second organic phase. In some embodiments, a carrier oil in a first organic phase can be any appropriate oil that can act as a shielding oil. Without being bound by any particular theory, it is believed that a shielding oil, e.g., in a first organic phase, can shield a hydrogel precursor against a carrier oil, e.g., in a second organic phase, that contains a cross-linker.

A surfactant can include any appropriate surfactant. In some embodiments, the surfactant in a first organic phase can be the same type of surfactant as the surfactant in a second organic phase. In some embodiments, a surfactant can be sorbitan monooleate (e.g., SPAN® 80). In some embodiments, a surfactant can be polysorbate 20 (e.g., TWEEN® 20). In some embodiments, a surfactant can be 4-(1,1,3,3-Tetramethylbutyl)phenyl-polyethylene glycol, t-Octylphenoxypolyethoxyethanol, Polyethylene glycol tert-octylphenyl ether (e.g., TRITON™ X-100). In some embodiments, a surfactant can be selected from the group consisting of sorbitan monooleate, polysorbate 20, and 4-(1,1,3,3-Tetramethylbutyl)phenyl-polyethylene glycol, t-Octylphenoxypolyethoxyethanol, Polyethylene glycol tert-octylphenyl ether). A surfactant can be present in any appropriate amount in a first organic phase. In some embodiments, a surfactant can be present in a first organic phase in an amount of about 0.2% to about 0.8% (v/v) (e.g., about 0.2% to about 0.4%, about 0.4% to about 0.6%, about 0.6% to about 0.8%, about 0.4% to about 0.8%, about 0.2%, about 0.3%, about 0.4%, about 0.5%, about 0.6%, about 0.7%, or about 0.8% (v/v)). A surfactant can be present in any appropriate amount in a second organic phase. In some embodiments, a surfactant can be present in a second organic phase in an amount of about 2% to about 5% (v/v) (e.g. about 2% to about 3%, about 2% to about 4%, about 3% to about 4%, about 3% to about 5%, about 4% to about 5%, about 2%, about 3%, about 4%, or about 5% (v/v)). In some embodiments, a second organic phase can include a cross-linker. A cross-linker can be any appropriate cross-linker. In some embodiments, a cross-linker can be a molecule that contains at least two thiol groups. In some embodiments, a cross-linker can be dithiothreitol (DTT). In some embodiments, a cross-linker can be octanedithiol. In some embodiments, a cross-linker can be DTT or octanedithiol. In some embodiments, a second organic phase can be an emulsion. In some embodiments, a cross-linker can be provided in the form of an aqueous phase of an emulsion. A cross-linker can be present in any appropriate concentration in an aqueous phase of an emulsion. In some embodiments, a cross-linker can be present in an aqueous phase of an emulsion in an amount of about 1 to about 30 mg/mL (e.g., 1 to about 2 mg/mL, about 1.5 to about 1.9 mg/mL, about 1 to about 10 mg/mL, about 10 to about 20 ng/mL, about 20 to about 22 mg/mL, about 22 to about 24 mg/mL, about 24 to about 26 mg/mL, about 26 to about 28 mg/mL, about 28 to about 30 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL). In some embodiments, a cross-linker can be present in an aqueous phase of an emulsion in an amount of about 20 to about 30 mg/mL. In some embodiments, a cross-linker can be present in an aqueous phase of an emulsion in an amount of about 25 mg/mL. In some embodiments, a cross-linker can be present in an aqueous phase of an emulsion in an amount such that the final concentration of cross-linker in the second organic phase is about 0.05 to about 2 mg/mL (e.g., about 1 to about 2 mg/mL, about 1.5 to about 1.9 mg/mL, 1.6 mg/mL, 1.7 mg/mL, 1.8 mg/mL, about 0.05 to about 0.15 mg/mL). In some embodiments, a cross-linker can be present in an aqueous phase of an emulsion in an amount such that the final concentration of cross-linker in the second organic phase is about 0.068 to about 0.136 mg/mL. An aqueous phase of an emulsion can be present in any appropriate ratio with an organic phase of the emulsion. In some embodiments, 1 part (by volume) of an aqueous phase can be present for every 10 to 20 parts (e.g., 10 to 12 parts, 12 to 14 parts, 14 to 16 parts, 16 to 18 parts, 18 to 20 parts, 10 parts, 11 parts, 12 parts, 13 parts, 14 parts, 15 parts, 16 parts, 17 parts, 18 parts, 19 parts, or 20 parts) of an organic phase. Such an emulsion can be made by any appropriate method (e.g., sonication).

In some aspects, any of the microcapsules as described herein can be fabricated by a method comprising mixing, to form a core-shell mixture, a core solution and a shell solution. In some aspects the core solution can comprise one or more analyte capture beads. In some aspects, the core solution can comprise one or more analyte detection beads. In some aspects, the core solution can comprise one or more analyte capture beads and one or more analyte detection beads. In some aspects, the shell solution can comprise a hydrogel precursor. The core-shell mixture can be pushed through an orifice into a first organic phase to form droplets of the core-shell mixture. The droplets can be passed into a second organic phase comprising a cross-linker. The shell solution can be cross-linked when in contact with the cross-linker in the second organic phase, thus forming a hydrogel sell around the core solution and thereby forming microcapsules. In some aspects, the method of fabricating the microcapsules can be performed in a microfluidic device.

For example, in some aspects, any of the microcapsules as described herein can be fabricated using a microfluidic device such as device 110 as shown in FIG. 4. A shell solution can be passed through at least a first channel 120A and a core solution can be passed through a second channel 130 into a common channel 140, thus forming a core-shell mixture in the common channel. In some embodiments, a shell solution can be passed through two or more channels (e.g., 120A and 120B). The aqueous core-shell mixture can be passed through an orifice 150 into a first chamber 160 filled with a first organic phase, resulting in formation of droplets that can, in some embodiments, retain their shape before cross-linking. Droplets can pass into a second chamber 170 filled with a second oil phase including a cross-linker. Upon contacting the cross-linker, hydrogel precursor molecules in the shell solution can become cross-linked and form the microcapsules 180.

Flow rates can be any appropriate flow rates. In some embodiments, a core solution can have a flow rate of about 3 μL/min to about 5 μL/min. In some embodiments, a shell solution can have a flow rate of about 3 μL/min to about 5 μL/min. In some embodiments, a first organic phase can have a flow rate of about 30 μL/min to about 40 μL/min. In some embodiments, a second organic phase can have a flow rate of about 40 μL/min to about 60 μL/min.

Formed microcapsules can be collect by methods known in the art and used in various detection assays. Microcapsules can be collected and stored (e.g., in 1% BSA in PBS). Microcapsules typically reside in an organic phase layer above an aqueous layer and slowly partition into the aqueous phase. Mechanical agitation can expedite the transfer of microcapsules from oil to aqueous phase.

Sensing capsules can be fabricated using droplet microfluidics to contain a hydrogel shell and an aqueous core with free-floating Ab-modified microbeads and/or nanobeads. The shell thickness can be controlled in part by the relative flow rates of core and shell solutions. Minimizing shell thickness can aid in rapid and unimpeded diffusion of cytokines and/or secondary Abs. A flow rate of 4 μl/min for both core and shell solutions can result in a hydrogel shell of ˜10 μm. In some embodiments, a flow rate of 4 μl/min can be chosen for a fabrication process. Despite a relatively thin shell, microcapsules can remain mechanically stable and resistant to breaking during a series of sequential vortex (˜250 rpm)/steady cycles.

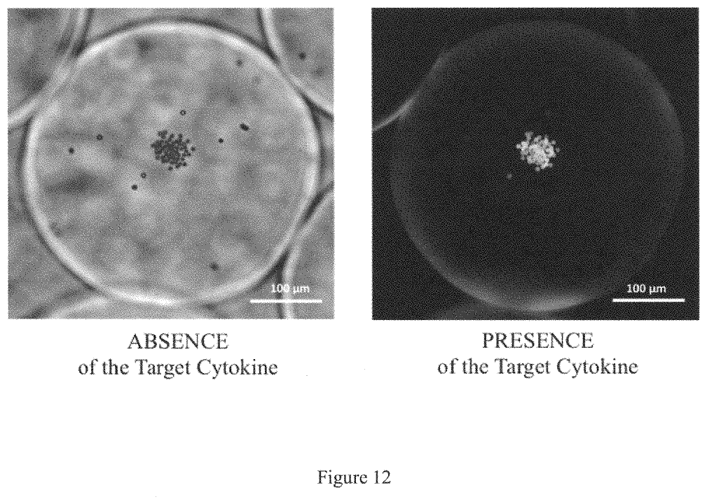

Fabricated microcapsules can be added to a biological sample to detect a target analyte. In some embodiments, a biological sample can be any of the biological samples described herein. In some embodiments, a biological sample is whole blood. In some embodiments, microcapsules can include a plurality of analyte capture beads. In some embodiments, microcapsules can include a plurality of analyte detection beads. In some embodiments, microcapsules can be incubated with a biological sample. An incubation can be any appropriate incubation. For example, an incubation can occur under cell culture conditions. In some embodiments, an incubation can be for about 1 hour to about 24 hours (e.g., about 1 hour to about 2 hours, about 1 hour to about 4 hours, about 1 hour to about 6 hours, about 1 hour to about 12 hours, about 1 hour to about 18 hours, about 2 hours to about 4 hours, about 2 hours to about 6 hours, about 2 hours to about 12 hours, about 2 hours to about 18 hours, about 2 hours to about 24 hours, about 4 hours to about 6 hours, about 4 hours to about 12 hours, about 4 hours to about 18 hours, about 4 hours to about 24 hours, about 6 hours to about 12 hours, about 6 hours to about 18 hours, about 6 hours to about 24 hours, about 12 hours to about 18 hours, about 12 hours to about 24 hours, about 18 hours to about 24 hours, about 1 hour, about 2 hours, about 4 hours, about 6 hours, about 12 hours, about 18 hours, or about 14 hours). In some embodiments, microcapsules can be removed from a biological sample following an incubation. Removal of microcapsules can be accomplished using any appropriate method. For example, microcapsules can be strained from a biological sample (e.g., using a strainer with 40-200 μm mesh size). In some embodiments, presence or absence of a target analyte can be detected using fluorescence microscopy. FIG. 12 is an image showing exemplary fluorescence microscopy when a target analyte is absent (left) and present (right). In some embodiments, a standard curve can be determined using known concentrations of a target analyte. In some such embodiments, the concentration of a target analyte in a biological sample can be quantified using a standard curve.

The materials and methods of the disclosure will be further described in the following Examples, which do not limit the scope the claims.

### EXAMPLES

### Example 1

### Fabrication of Microfluidic Devices

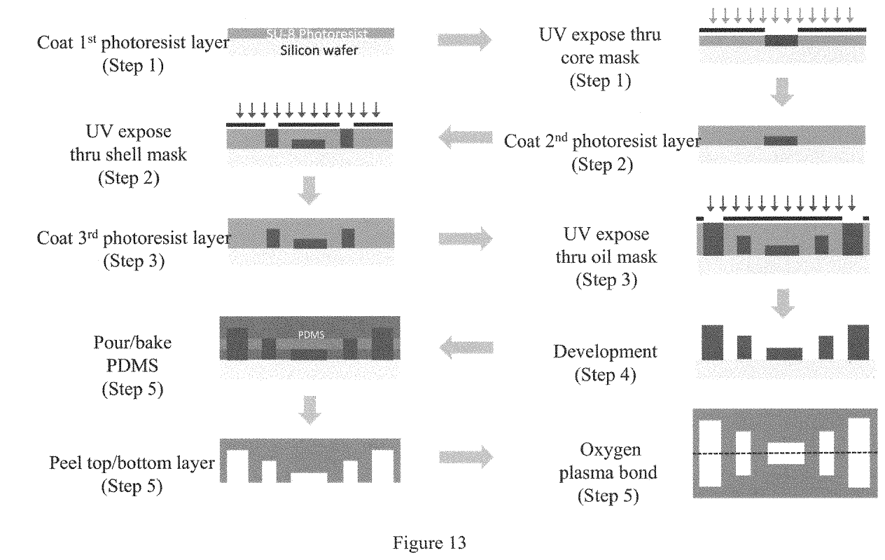

Droplet generating devices were fabricated as described in Siltanen C, Diakatou M Lowen J, Haque A, Rahimian A, Stybayeva G and Revzin A. 2017. One step fabrication of hydrogel microcapsules with hollow core for assembly and cultivation of hepatocyte spheroids. Acta Biomater 50:428-36. Briefly, top and bottom masters were prepared by spin coating three layers of SU-8 photoresist (MicroChem, Westborough, Mass.) on a 4-inch silicon wafer (University Wafer, USA). The first layer (60 μm) was exposed to UV light through a photomask repre-senting the core channel pattern. After post-exposure bake, a second SU-8 layer (40 μm) was similarly spin coated and exposed through a second photomask corresponding to the shell channel pattern using a mask aligner (Kloe UV-KUB 3, France). The spin-coating and exposure steps were repeated for the oil channel layer (50 μm). Subsequently, a wafer was placed into a developer solution to remove photoresist regions not exposed to UV light. The process of three spin-coating and exposure steps followed by a single development step was better than developing each layer, because cleaning the surface in preparation for the next layer could damage the former layer. PDMS (Ellsworth adhesive, Minneapolis, Minn.) was then poured over a silicon master and replica molded by standard soft lithography procedures. Cured “top” and “bottom” PDMS pieces were then treated with 40 s of exposure to air plasma (G-500 plasma cleaning system, Yield Engineering Systems, Liver-more, CA), and manually aligned under a stereoscope (Zeiss Stemi 508, Germany) using deionized water as a lubricating layer. Aligned chips were then placed on a hotplate at 95° C. for 4 h to remove the water layer and bond the two PDMS layers together. Subsequently, microfluidic devices were infused with an Aquapel solution to render surfaces hydrophobic in preparation for droplet generation. The final chip dimensions (height) were a 120 μm oil channel. A schematic of an exemplary device is shown in FIGS. 3 and 4. FIG. 13 is a flow chart of an exemplary device fabrication process.

### Example 2

### Microfluidic Device Operation and Capsule Fabrication

Microfluidic coaxial flow-focusing devices were infused with 4 different solutions to generate capsules: 1) a core solution composed of 8% w/v PEG (35 kDa) and 17% Optiprep densifier (Sigma-Aldrich, St. Louis, Mo.) dissolved in desired cell culture media containing capture beads, 2) a shell solution containing 4-8% w/v 4-arm maleimide functionalized polyethylene glycol (PEG4MAL, Creative PEGwork, Durham, N.C.) and 15 mM triethanolamine (TEA) dis-solved in the same media, 3) a shielding organic phase of mineral oil with 0.5% v/v Span-80 surfactant, and 4) a crosslinking organic phase of mineral oil with 3% Span-80 and a 1:15 emulsion of 25 mg/mL dithiothrietol (DTT) (Sigma-Aldrich, St. Louis, Mo.) dissolved in DI water (emulsion was generated by sonicating oil/water mixture for 30 min in an ultrasonic bath). Each of the 4 solutions was filtered through 0.2 μm centrifuge filters and loaded into syringes under sterile conditions. Following this sterilization step, core, shell, shielding oil, and crosslinking solutions were infused into separate inlets (see FIG. 4) of a microfluidic device using syringe pumps (Harvard Apparatus, Holliston, Mass.) at flow rates of 3-5 μL/min for core, 3-5 pL/min for shell, 30-40 μL/min for shielding oil, and 40-60 μL/min for crosslinking oil. Pushing the aqueous mixture of core and shell solutions through an orifice into the oil phase resulted in formation of droplets that retained their shape prior to gelation. Upon addition of the dithiolated cross-linker (DTT) through the fourth stream, PEG4Mal molecules in the shell became cross-linked forming a gel layer. These capsules were collected into an Eppendorf tube containing 1% BSA in PBS. The capsules resided in the oil phase layer above the aqueous layer in the tube and slowly partition into the liquid phase. Mechanical agitation expedited the transfer of capsules from oil to aqueous phase.

### Example 2

### Microfluidic Device Operation and Capsule Fabrication

Microfluidic coaxial flow-focusing devices are infused with 4 different solutions to generate capsules: 1) a core solution composed of 8% w/v PEG (35 kDa) and 17% Optiprep densifier (Sigma-Aldrich, St. Louis, Mo.) dissolved in desired cell culture media containing capture beads and detection beads, 2) a shell solution containing 4-8% w/v 4-arm maleimide functionalized polyethylene glycol (PEG4MAL, Creative PEGwork, Durham, N.C.) and 15 mM triethanolamine (TEA) dis-solved in the same media, 3) a shielding organic phase of mineral oil with 0.5% v/v Span-80 surfactant, and 4) a crosslinking organic phase of mineral oil with 3% Span-80 and a 1:15 emulsion of 25 mg/mL dithiothrietol (DTT) (Sigma-Aldrich, St. Louis, Mo.) dissolved in DI water (emulsion is generated by sonicating oil/water mixture for 30 min in an ultrasonic bath). Each of the 4 solutions is filtered through 0.2 μm centrifuge filters and loaded into syringes under sterile conditions. Following this sterilization step, core, shell, shielding oil, and crosslinking solutions are infused into separate inlets (see FIG. 4) of a microfluidic device using syringe pumps (Harvard Apparatus, Holliston, Mass.) at flow rates of 3-5 μL/min for core, 3-5 μL/min for shell, 30-40 μL/min for shielding oil, and 40-60 μL/min for crosslinking oil. Pushing the aqueous mixture of core and shell solutions through an orifice into the oil phase results in formation of droplets that retained their shape prior to gelation. Upon addition of the di-thiolated cross-linker (DTT) through the fourth stream, PEG4Mal molecules in the shell become cross-linked forming a gel layer. These capsules are collected into an Eppendorf tube containing 1% BSA in PBS. The capsules reside in the oil phase layer above the aqueous layer in the tube and slowly partition into the liquid phase. Mechanical agitation expedites the transfer of capsules from oil to aqueous phase.

### Example 3

### Preparation of Antibody-Functionalized Capture Microbeads

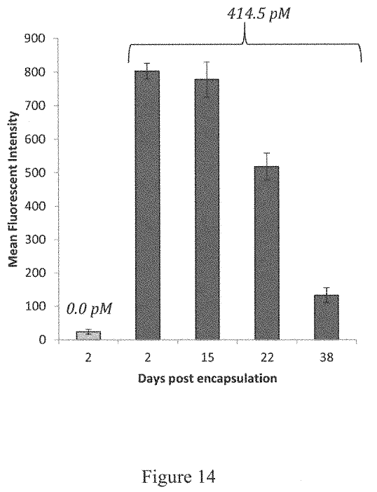

Streptavidin-coated microbeads (Spherotech, Lake Forest, Ill.) were used for capture of cytokines in our experiments. Biotinylated anti-IFN-γ and anti-TNF-α Abs were immobilized on microbeads (5- 5.9 μm diameter) according to protocols described in Son K, Rahimian A, Shin D, Siltanen C, Patel T, Revzin A. 2016. Microfluidic compartments with sensing microbeads for dynamic monitoring of cytokine and exosome release from single cells. Analyst 141:679-88 and Son K J, Gheibi P, Stybayeva G, Rahimian A, Revzin A. 2017. Detecting cell-secreted growth factors in microfluidic devices using bead-based biosensors. Microsyst Nanoeng 3. Briefly, prior to functionalization, microbeads were incubated with Pierce protein-free blocking buffer (ThermoFisher Scientific, Grand Island, N.Y.) for 2h at 4° C., then washed with PBS via a centrifugation (13,000 rcf, 5 min). In the next step, 50 μL of streptavidin-microbeads (˜3.6×10⁶ beads) was incubated with 5 μg of biotinylated capture Abs (R&D systems, Minneapolis, Minn.) in 250 μL 1% BSA in PBS overnight at 4° C. After Ab immobilization, microbeads were washed in PBS by centrifugation before reconstituting in 2× concentrated core solution. The microbeads were encapsulated according to the protocol described above. Encapsulated microbeads were stable at 4° C. in the dark for up to two weeks (See FIG. 14).

### Example 4

### Preparation of Antibody-Functionalized Detection Microbeads

Streptavidin-coated fluorescent polystyrene particles (diameter=0.4-0.6 μm; Nile Red and Yellow) are purchased from Spherotech (Lake Forest, Ill., USA). The beads are streptavidin coated by the manufacturer and were incubated with biotinylated anti-GF Abs as follows. First, microbeads were washed with PBS three times using a centrifugation/washing protocol (12 000 r.c.f. for 3 min, Centrifuge 5424, Eppendorf, Hamburg, Germany). Detection microbeads (˜1.4×106 beads or 0.1 mg) are then incubated overnight at 4° C. with 4 μg of biotinylated Abs in 50 μL of PBS solution containing 1% bovine serum albumin (BSA). Five micrograms of streptavidin-coated fluorescent microbeads (˜2.0×10⁸ beads) are incubated overnight at 4° C. with 2 μg of biotinylated Abs in 50 μL of PBS containing 1% BSA. The detection microbeads functionalized with Abs are washed with PBS three times, followed by blocking with 2% BSA for 30 min at room temperature. Ab microbeads are stored for up to 2 weeks at 4° C.

### Example 5

### Detecting TNF-α and IFN-α Using Encapsulated Capture Microbeads

A sandwich immunoassay was used to detect capture of cytokine molecules on capture microbeads. Secondary Abs, polyclonal human anti-IFN-γ and anti-TNF-α (R&D systems, Minneapolis, Minn.), were labeled with Alexa-488 and Alexa-546 respectively using commercial kits and following manufacturer's instructions (ThermoFisher Scientific, Grand Island, N.Y.).

For microcapsule visualization, 1 μg/mL of thiolated dye (Rhoda-min/FITC/Cy5)/PEG conjugate (SH-PEG-dye 5 kDa) (Creative PEGwork, Durham, N.C.) was included in the shell solution along with 15 mM TEA. The latter chemical was used to reduce thiol groups and enhance uniformity of dye conjugation within the gel.

In order to establish figures of merit (limit of detection and linear range), encapsulated microbeads were challenged with different concentrations of recombinant IFN-γ and TNF-α (R&D systems, Minneapolis, Minn.). After 2 h incubation with recombinant cytokines, microcapsules were labeled by 2 h exposure to fluorescently labeled secondary Abs and then imaged with fluorescence microscope (Olympus IX 83, Tokyo, Japan). Calibration curves of fluorescence intensity vs. cytokine concentration were constructed with the aid of image analysis software (ImageJ2 ver. 1.51u).

### Example 6

### Detecting TNF-α and IFN-α Using Encapsulated Capture and Detection Microbeads

Fluorescence microscopy is used used to detect capture of cytokine molecules on capture microbeads.

For microcapsule visualization, 1 μg/mL of thiolated dye (Rhoda-min/FITC/Cy5)/PEG conjugate (SH-PEG-dye 5 kDa) (Creative PEGwork, Durham, N.C.) is included in the shell solution along with 15 mM TEA. The latter chemical is used to reduce thiol groups and enhance uniformity of dye conjugation within the gel.

In order to establish figures of merit (limit of detection and linear range), encapsulated microbeads including fluorescently labeled detection microbeads in addition to capture microbeads are challenged with different concentrations of recombinant IFN-γ and TNF-α (R&D systems, Minneapolis, Minn.). After 2 h incubation with recombinant cytokines, microcapsules are imaged with fluorescence microscope (Olympus IX 83, Tokyo, Japan). Calibration curves of fluorescence intensity vs. cytokine concentration are constructed with the aid of image analysis software (ImageJ2 ver. 1.51u)

### Example 7

### Blood Sample Collection and Processing

The study was approved by the Mayo Clinic Institutional Review Board (IRB#: 09-003253). All study participants signed an informed written consent and were enrolled between August 2017 and December 2017. Study subjects included unexposed individuals with negative QuantiFERON-TB Gold In-Tube (QFT) results and subjects with LTBI diagnosis as per current guidelines criteria, including asymptomatic subjects at risk of prior tuberculosis (TB) exposure, negative chest X-rays, and by prior positive QFT results as previously described (Escalante P, Peikert T, Van Keulen V P, Erskine C L, Bornhorst C L, Andrist B R, McCoy K, Pease L R, Abraham R S, Knutson K L, Kita H, Schrum A G, and Limer A H. 2015. Combinatorial Immunoprofiling in Latent Tuberculosis Infection. Toward Better Risk Stratification. Am J Respir Crit Care Med 192:605-17 and Lewinsohn D M, Leonard M K, LoBue P A, Cohn D L, Daley C L, Desmond E, Keane J, Lewinshon D A, Loeffler A M, Mazurek G H, O'Brien R J, Pai M, Richeldi L, Salfinger M, Shinnick T M, Sterling T R, Warshauer D M, Woods G L. 2017. Official American Thoracic Society/Infectious Diseases Society of America/Centers for Disease Control and Prevention Clinical Practice Guidelines: Diagnosis of Tuberculosis in Adults and Children. Clin Infect Dis 64:111-5). Each blood sample was collected into three lml QFT tubes (Qiagen, Germantown, Md.) labeled “Mitogen”, “Antigen”, and “Nil”, representing the positive control, antigen peptide mixture, and negative control samples, respectively. QFT was performed as recommended by the manufacturer. A cut-off level of IFN-γ ≥0.35 IU/ml and >50% above nil defined a QFT(+) test. Research laboratory technicians were blinded to the results of QFT. Approximately 125 sensing microcapsules were added into each tube and were incubated overnight at 37° C. with 5% CO₂ on a shaker operating at 131 rpm. After incubation in blood, microcapsules were retrieved using 37 μm cell strainers (Stemcell technologies, Vancouver, Canada) and were then stained with fluorescently-labeled detection Abs for 2 h at room temperature. After staining, microcapsules were washed with 1% BSA in PBS and visualized using a fluorescence microscope (Olympus IX 83, Tokyo, Japan). On-bead fluorescence intensity was analyzed using ImageJ2 software.

Peripheral blood mononuclear cells (PBMCs) were also used in some of the calibration experiments. These cells were isolated in the Mayo Clinic transfusion medicine component laboratory as reported in Dietz A B, Bulur P A, Emery R L, Winters J L, Epps D E, Zubair A C and Vuk-Pavolovic S. 2006. A novel source of viable peripheral blood mononuclear cells from leukoreduction system chambers. Transfusion 46:2083-9. PMA (phorbol 12-myristate-13-acetate) and ionomycin (Sigma-Aldrich, St. Louis, Mo.) stimulation was carried out using 100 ng/ml PMA and 1 μg/mL ionomycin.

### Example 8

### Blood Sample Collection and Processing

The study is approved by the Mayo Clinic Institutional Review Board. All study participants sign an informed written consent. Study subjects include unexposed individuals with negative QuantiFERON-TB Gold In-Tube (QFT) results and subjects with LTBI diagnosis as per current guidelines criteria, including asymptomatic subjects at risk of prior tuberculosis (TB) exposure, negative chest X-rays, and by prior positive QFT results as in Example 7. Each blood sample is collected into three lml QFT tubes (Qiagen, Germantown, Md.) labeled “Mitogen”, “Antigen”, and “Nil”, representing the positive control, antigen peptide mixture, and negative control samples, respectively. QFT is performed as recommended by the manufacturer. A cut-off level of IFN-γ≥0.35 IU/ml and >50% above nil defined a QFT(+) test. Research laboratory technicians are blinded to the results of QFT. Approximately 125 sensing microcapsules (including both analyte capture beads and analyte detection beads) are added into each tube and were incubated overnight at 37° C. with 5% CO₂ on a shaker operating at 131 rpm. After incubation in blood, microcapsules are retrieved using 37 μm cell strainers (Stemcell technologies, Vancouver, Canada) and are then washed with 1% BSA in PBS and visualized using a fluorescence microscope (Olympus IX 83, Tokyo, Japan). On-bead fluorescence intensity is analyzed using ImageJ2 software.

Peripheral blood mononuclear cells (PBMCs) are also used in some of the calibration experiments. These cells are isolated in the Mayo Clinic transfusion medicine component laboratory as reported in Dietz A B, Bulur P A, Emery R L, Winters J L, Epps D E, Zubair A C and Vuk-Pavolovic S. 2006. A novel source of viable peripheral blood mononuclear cells from leukoreduction system chambers. Transfusion 46:2083-9. PMA (phorbol 12-myristate-13-acetate) and ionomycin (Sigma-Aldrich, St. Louis, Mo.) stimulation is carried out using 100 ng/ml PMA and 1 μg/mL ionomycin.

### Example 9

### Statistical Analysis

Data are represented as mean ± SEM. Statistical significance be-tween experimental groups was assessed using a two-tailed student's t-test and p-values <0.05 or in some cases <0.01 were considered statistically significant.

### Example 10

### Diffusion of Cytokines

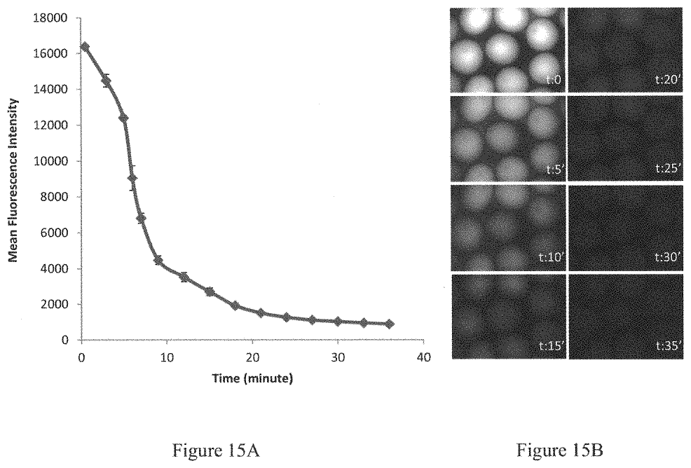

Additionally, in order to investigate the diffusion of secreted cytokines through the shell and into the core where the capture beads were situated, microcapsules were loaded with fluorescent molecules (20 kDa Dextran-Alexa-546) similar in the size to IFN-γ and TNF-α and fluorescence was monitored over time. As can be seen from FIG. 15, after ˜40 min, fluorescence signals inside and outside capsules became similar. These experiments demonstrated that porosity of the hydrogel shell of microcapsules was sufficient for diffusion of cytokines.

### Example 11

### Optimizing Microcapsule Numbers to Ensure Sensitive Detection of Cytokines

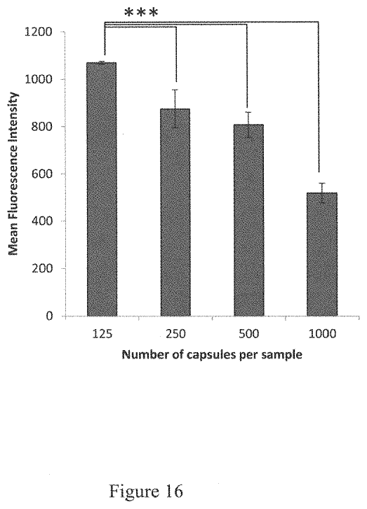

Fluorescence signal in microcapsules may depend not only on the concentration of cytokines in the sample but also on the number of Ab-binding sites in the sample. There are different strategies for controlling the number of Ab sites 1) by adjusting Ab concentration during bead functionalization, 2) by controlling the number of encapsulated capture microbeads, and 3) by controlling the number of microcapsules present in the sample. Maximizing the number of Ab molecules per bead was chosen to increase the possibility of target analyte capture. In parallel, the number of sensing beads per capsule was selected by running a series of experiments with different population sizes of the beads. Considering that the range of Poisson-distributed beads varied by about ±20 beads per capsule, about 50 beads per capsule was a condition that would result in a sufficient number of beads for analysis of cytokine binding. Scenario 3—optimizing the number of capsules per sample was chosen as the variable to optimize. Different numbers of microcapsules (ranging from 125 to 1000) were placed into 1 mL of 1% BSA solution containing 14 pM concentration of IFN-γ for 16 h and then stained with detection Abs. Results shown in FIG. 16 reveal that the average fluorescence from a sample containing 125 microcapsules was significantly higher than the intensity from samples containing a larger number of capsules. Therefore, about 100 microcapsules per 1 mL sample was chosen as a target. Using fewer than 100 microcapsules was impractical, since it was difficult to isolate/filter a small number capsules prior to staining with secondary Abs and fluorescence detection.

### Example 12

### Effect of DTT

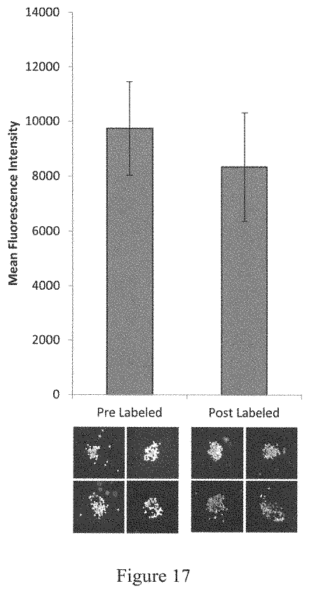

DTT, the cross linker used to create a hydrogel shell during the droplet fabrication process, has been reported to affect functionality of Abs (Hutterer K M, Hong R W, Lull J, Zhao X Y, Wang T, Pei R, Le M E, Borisov O, Pier R, Liu Y D, Petty K, Apostol I, and Flynn G C. 2013. Monoclonal antibody disulfide reduction during manufacturing Untangling process effects from product effects. Mabs-Austin 5:608-13 and Crivianu-Gaita V, Romaschin A, Thompson M. 2015. High efficiency reduction capability for the formation of Fab antibody fragments from F(ab)2 units. Biochem Biophys Rep 2:23-8). To check whether Abs immobilized on capture microbeads was affected by this reagent, two populations of microcapsules were compared: 1) microcapsules with Ab-functionalized microbeads exposed to DTT during microfluidic fabrication, and 2) microcapsules with avidin-functionalized microbeads that were exposed to biotinylated Abs post encapsulation. As shown by data in FIG. 17, both populations of microcapsules exhibited similar fluorescence intensity after challenge with 57.5 μM (1 ng/mL) of TNF-α. This suggested that microfluidic fabrication of capsules did not affect functionality of capture Abs.

### Example 13

### Characterizing Responses of Microcapsules to Known Concentrations of IFN-γ and TNF-α

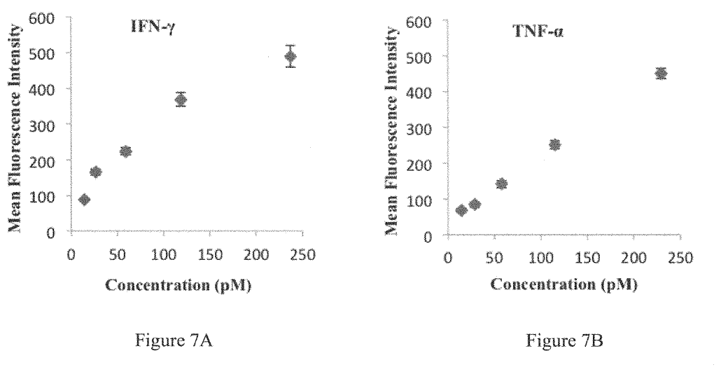

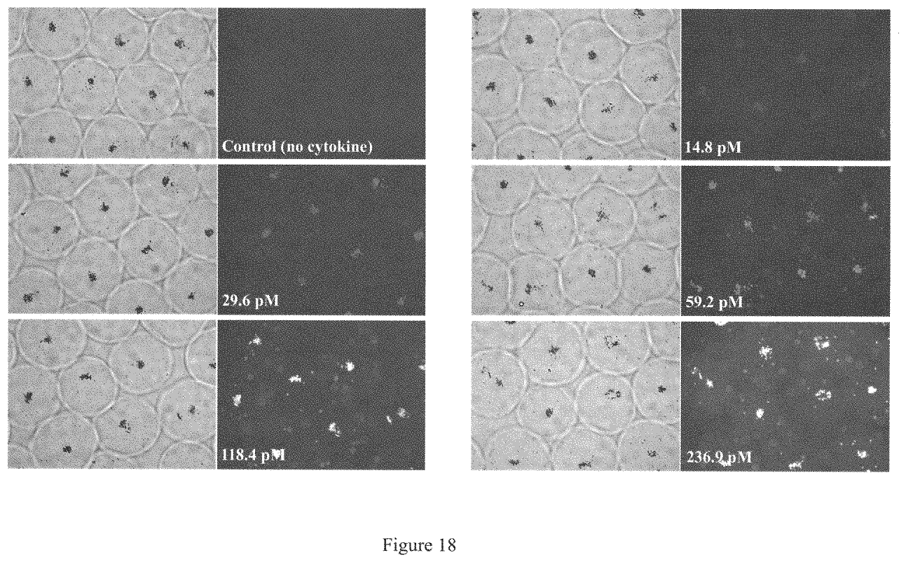

Calibration curves were constructed to establish a relationship between fluorescence intensity and cytokine concentrations. For these experiments, microcapsules were placed into test solutions containing different concentrations of IFN-γ or TNF-α, in the range of 0 to 250 μM. This concentration range was selected based on the reported levels of these cytokines in blood (Liu Y, Rahimian A, Krylyuk S, Vu T, Crulhas B, Stybayeva G, Imanbekdova M, Shin D S, Davydov A and Revzin A. 2017. Nanowire Aptasensors for Electrochemical Detection of Cell-Secreted Cytokines. ACS Sens 2:1644-52). Staining with fluorescently-labeled detection Abs revealed that on-bead fluorescence signal varied depending on the concentration of recombinant cytokines (see FIG. 18). Calibration curves presented in FIG. 7A and FIG. 7B demonstrate that the response in sensing microcapsules remained linear for the concentration range tested. FIG. 7A is a calibration curve constructed by exposure of sensing microcapsules to concentrations of IFN-γ varying from 14 μM (250 pg/ml) to 236 pM (4 ng/ml). FIG. 7B is a calibration curve for TNF-α constructed by exposure of sensing microcapsules to concentrations ranging from 14 pM to 230 pM (250 pg/ml to 4 ng/ml). Both calibration curves were constructed by measuring mean fluorescent intensity of the encapsulated beads after 120 minutes of incubation with known concentrations of analyte (n=12, p<0.05). This demonstrates that fluorescence intensity associated with the lowest concentration of IFN-γ and TNF-α tested, 14 μM, was significantly higher than the control sample without cytokine (P value<0.05). This is comparable to other previously reported fluorescence immuno-assays without signal amplification (Giljohann D A, Mirkin C A. 2009. Drivers of biodiagnostic development. Nature 462:461-4, Yager P, Edwards T, Fu E, Helton K, Nelson K, TAM M R and Weigl B H. 2006. Microfluidic diagnostic technologies for global public health. Nature 442:412-8, and Liangcheng Zhou F D, Hao Chen, Wei Ding, Weihua Zhang, and Stephen Y. Chou. 2012. Enhancement of Immunoassay's Fluorescence and Detection Sensitivity Using Three-Dimensional Plasmonic Nano-Antenna-Dots Array. Anal Chem 84 4489-95). Another commonly used method is to set a limit of detection as a signal 3× greater than noise. Based on this method, the limit of detection is ˜9 and ˜5 pM for IFN-γ and TNF-α, respectively.

### Example 14

### Performance in a Fouling Environment

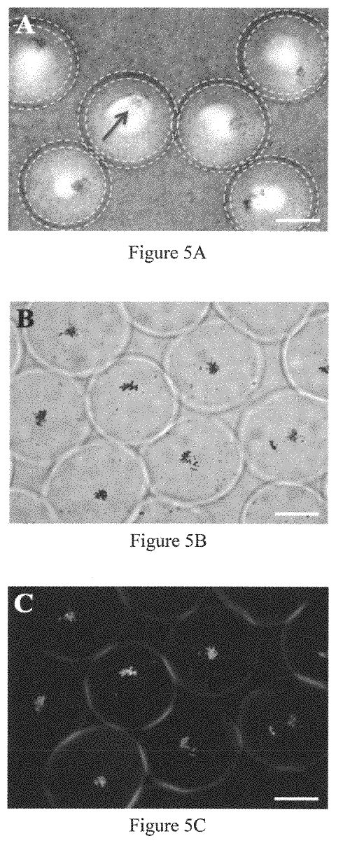

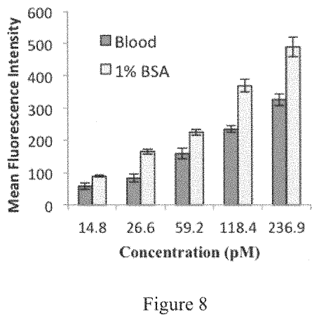

In order to verify the performance of the bio-sensor in a fouling environment, the capture microcapsules were incubated overnight in blood with spiked concentrations of recombinant INF-γ. As shown in (FIG. 8), fluorescence signal from sensing microbeads correlated with different concentrations of target cytokine. FIG. 8 is illustrates the detection of recombinant IFN-γ in 1% BSA (bovine serum albumin) in PBS) as well as in whole blood after 16 h incubation at 37° C. with 5% CO₂. Concentration range tested—from 14 to 236 pM. The difference (25-30%) in signals obtained from 1% BSA solution and whole blood may be attributed to a much more complex composition of the latter. Leukocytes in blood may uptake cytokines including IFN-γ, or there may also be proteases in blood that digest cytokines contributing to a lower detected signal. It is noted that microcapsules extracted from blood after overnight incubation at 37° C. remained free of leukocytes (see FIG. 5A, B). Therefore, leukocyte adhesion did not result in a barrier to cytokine transport and did not contribute to lower signals detected in blood (FIG. 5C). FIG. 5A represents the microcapsules in a fouling environment (whole blood). The yellow dotted double line indicates the shell of microcapsules. The arrow points to the beads that are settled in the core. FIG. 5B shows the recovered microcapsules after overnight incubation with stimulated blood at 37° C. FIG. 5C demonstrates the INF-γ signal on ca beads (green) in the core of the same capsules with Rhoda-mine labeled hydrogel shell (red). The scale bar is 200 μm.

### Example 15

### Simultaneous Detection of IFN-γ and TNF-α in the Same Sample

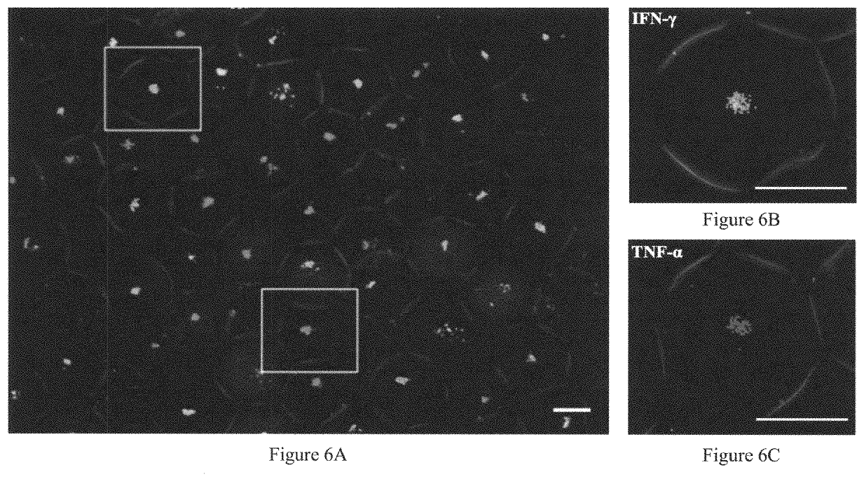

Microcapsule-based immunoassays are amenable to multiplexed detection of cytokines. Several routes for multiplexing can be envisioned, for example, by labeling the hydrogel shell with different fluorescent dyes or by utilizing detection Abs with different fluorophore labels. For a proof-of-concept experiment, two populations of microcapsules were, one containing anti-TNF-α microbeads and the second containing anti-IFN-γ beads, and then placed microcapsules into a test sample containing 60 pM of IFN-γ and TNF-α. Staining with detection Abs, anti-IFN-γ Alexa 488 (green), and anti-TNF-α Alexa 546 (red) revealed green and red fluorescence signal from IFN-γ and TNF-α sensing microcapsules, respectively (FIG. 6). Using calibration curves described in FIG. 7, fluorescence intensity was converted into concentration, 62.32 pM of IFN-γ and 57.01 pM of TNF-α. These values were similar to the initial concentration of cytokines spiked into the solution. FIG. 6 demonstrates dual cytokine detection using a mixed population of microcapsules containing IFN-γ and TNF-α specific microbeads. Microcapsules were incubated with a mixture of 60 pM IFN-γ and TNF-α and then exposed to anti-IFN-γ Alexa 488 (green) and anti-TNF-α Alexa 546 (red). The scale bar is 200 μm.

### Example 16

### Investigation of Cross-Reactivity

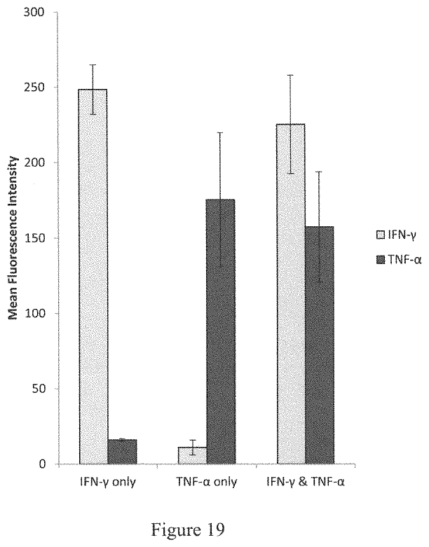

The performance of microcapsules for detection of IFN-γ and TNF-α was also in buffers containing one or both of these cytokines. FIG. 19 highlights the fact that a fluorescence signal was observed from the correct set of micro-capsules in the presence of the correct cytokine. No cross-reactivity between Ab-modified microbeads or off-target capture of incorrect cytokines was observed in the microcapsules.

### Example 17

### Comparison of the Sensing Microcapsules with ELISA from the IGRA Method for the Diagnosis of LTBI

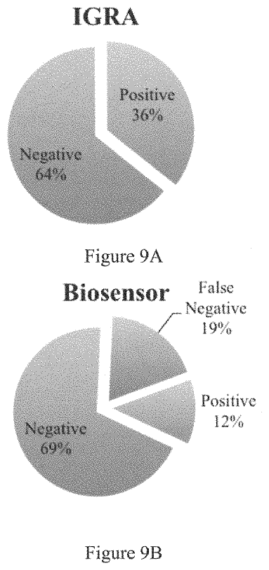

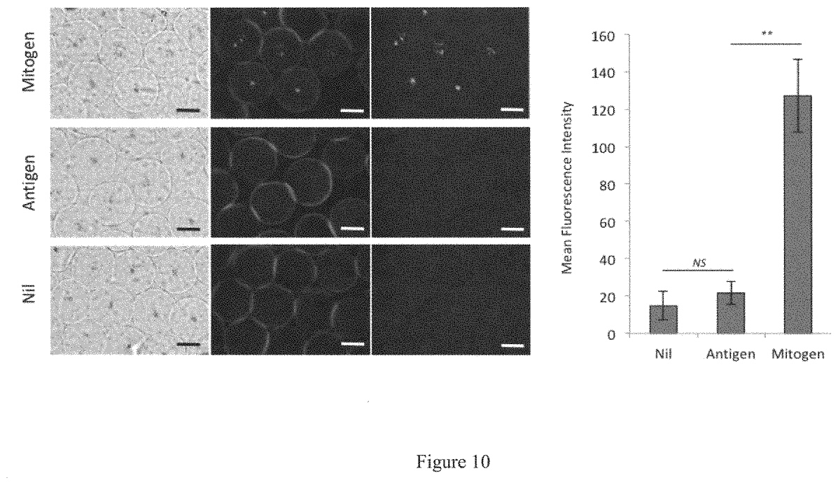

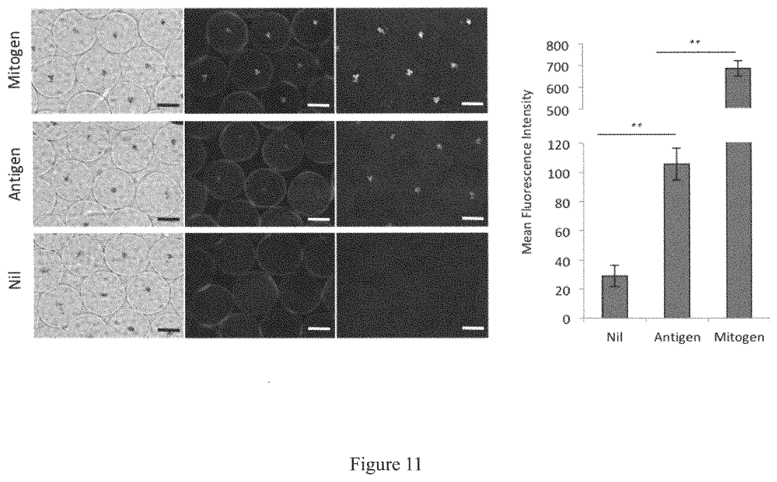

In order to further validate this technology, the sensing microcapsules were compared with the QFT method to detect IFN-y in whole blood samples from 5 QFT(+) patients with LTBI diagnosis and 9 unexposed controls. To that end, the QFT platform was utilized to obtain whole blood samples incubated with the specific M. tuberculosis antigen peptide mixtures and controls to compare the utility of the sensing micro-capsules to detect IFN-γ levels and utilized the ELISA method of the QFT assay as a gold standard comparison. The workflow of this experiment is shown in FIG. 2. Microcapsules were placed into QFT tubes marked as Mitogen, Antigen, or Nil were incubated in whole unprocessed blood. Stimulation of leukocytes and production of IFN-γ was expected to occur in the tubes during the 16 h incubation. Microcapsules were then recovered and characterized using fluorescence microscopy. Limited to no cell attachment to microcapsules was observed, suggesting that PEG hydrogel shells were effective in eliminating fouling (Banerjee I, Pangule R C, Kane R S. 2011. Antifouling Coatings: Recent Developments in the Design of Surfaces That Prevent Fouling by Proteins, Bacteria, and Marine Organisms. Adv Mater 23:690-718). An analysis of 14 study subject samples revealed that the technology was accurate in delivering correct diagnosis in 11 out of 14 cases (see Table 1 and FIGS. 9A and 9B). To achieve diagnosis for the sensing microcapsules, criteria for positive, negative, and intermediate results as outlined in Table 2 was used. FIGS. 10 and 11 show representative signals from microcapsules incubated with QFT (+) and QFT (−) samples. FIG. 10 shows a sample that that tested negative for LTBI, FIG. 11 shows a sample that tested positive for positive for LTBI. The shell of the capsules is labeled with Rhodamin (red fluorescent), and anti-IFN-γ is conjugated to lexa-488 (green). Green fluorescence indicates presence of IFN-γ in the blood sample. The fluorescence images converted into binary (black & white) and MFIs were measured using Image J (n=12, **p<0.01). The scale bar is 200 μm. LTBI, Late Tuberculosis Infection; IFN-γ, Interferon-γ; MFI, Mean Fluorescence Intensity; Mitogen, Positive Control; Anti-gen, Test; Nil, Negative Control.

It is noted that all three cases of incorrect diagnosis in this Example were false negatives in samples with low levels of IFN-γ (<10 pM). Despite somewhat limited sensitivity for detecting LTBI with low levels of IFN-γ, the concept of encapsulated immunoassays holds significant promise for sensing/sampling composition of complex bio-logical fluids such as blood.

TABLE 1

| Case # | IGRA | Biosensor |
|---|---|---|
| 1 | Negative | Negative |
| 2 | Positive | Negative |
| 3 | Negative | Negative |
| 4 | Positive | Positive |
| 5 | Negative | Negative |
| 6 | Negative | Negative |
| 7 | Negative | Negative |
| 8 | Negative | Negative |
| 9 | Negative | Negative |
| 10 | Positive | Negative |
| 11 | Negative | Negative |
| 12 | Negative | Negative |
| 13 | Positive | Intermediate |
| 14 | Positive | Positive |

TABLE 2
Interpretation of results. Nil, Negative Control; Ag, Antigen; Mtg, Mitogen;
MFI, Mean Fluorescent Intensity; M. TB, Mycobacterium Tuberculosis

| | Difference between | Mtg | Difference between | | |
|---|---|---|---|---|---|
| Nil (MFI) | Nil & Ag (MFI) | (MFI) | Ag & Mtg (MFI) | Test Result | Interpretation |
| <40% of Mtg | Non-Significant | >70 | Significant | Negative | M. Tuberculosis NOT likely |
| | (P value > 0.05) | | (P value < 0.05) | | |
| <40% of Mtg | Significant | >70 | Significant | Positive | M. Tuberculosis likely |
| | (P value < 0.05) | | (P value < 0.05) | | |
| <40% of Mtg | Significant | <70 | Any | Intermediate | Results are intermediate for |
| | (P value < 0.05) | | | | TB antigen responsiveness |
| >40% of Mtg | Any | Any | Any | Intermediate | Results are intermediate for |
| | | | | | TB antigen responsiveness |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A microcapsule comprising:
an aqueous core comprising an analyte capture bead and an analyte detection bead; and
a hydrogel shell.

2. The microcapsule of claim 1, wherein the analyte capture bead is a microbead comprising an analyte-specific binding moiety.

3. The microcapsule of claim 2, wherein the analyte-specific binding moiety is an analyte-specific antibody.

4. The microcapsule of claim 1, wherein the analyte detection bead is a nanobead comprising an analyte-specific binding moiety and a detection moiety.

5. The microcapsule of claim 4, wherein the detection moiety is a fluorophore.

6. The microcapsule of claim 4, wherein the detection moiety is a fluorescent dye.

7. The microcapsule of claim 1, wherein the analyte detection bead is a fluorescent nanobead conjugated to an analyte-specific antibody.

8. The microcapsule of claim 1, wherein the aqueous core further comprises a polymer.

9. The microcapsule of claim 1, wherein the aqueous core further comprises a densifier.

10. The microcapsule of claim 1, wherein the hydrogel shell comprises a cross-linked PEG hydrogel.

11. The microcapsule of claim 1, wherein the hydrogel shell further comprises a cell-specific capture moiety.

12. The microcapsule of claim 11, wherein the cell-specific capture moiety comprises an antibody to a cell surface molecule.

13. The microcapsule of claim 1, wherein the hydrogel shell further comprises one or more magnetic nanoparticles.

14. A composition comprising the microcapsule of claim 1.

15. A method of making the microcapsule of claim 1, comprising:
mixing, to form a core-shell mixture, a core solution comprising one or more analyte capture beads and one or more analyte detection beads, and a shell solution comprising a hydrogel precursor;
pushing the core-shell mixture through an orifice into a first organic phase to form droplets of the core-shell mixture;
passing the droplets into a second organic phase comprising a cross-linker;
cross-linking the hydrogel precursor to form microcapsules having hydrogel shells; and
collecting the microcapsules.

16. The method of claim 15, wherein the method is performed in a microfluidic device.

17. The method of claim 15, wherein the first organic phase comprises a first carrier oil and a surfactant.

18. The method of claim 17, wherein the first carrier oil is selected from the group consisting of mineral oil, a fluorinated oil, and mixtures thereof.

19. The method of claim 18, wherein the first carrier oil is mineral oil.

20. The method of claim 17, wherein the surfactant is selected from the group consisting of sorbitan monooleate, polysorbate 20, and 4-(1,1,3,3-Tetramethylbutyl)phenyl-polyethylene glycol, t-Octylphenoxypolyethoxyethanol, Polyethylene glycol tert-octylphenyl ether, and mixtures thereof.

21. The method of claim 20, wherein the surfactant is sorbitan monooleate.

22. The method of claim 15, wherein the second organic phase further comprises a second carrier oil and a surfactant.

23. The method of claim 22, wherein the second carrier oil is selected from the group consisting of mineral oil, a fluorinated oil, and mixtures thereof.

24. The method of claim 23, wherein the second carrier oil is mineral oil.

25. The method of claim 22, wherein the surfactant is selected from the group consisting of sorbitan monooleate, polysorbate 20, and 4-(1,1,3,3-Tetramethylbutyl)phenyl-polyethylene glycol, t-Octylphenoxypolyethoxyethanol, Polyethylene glycol tert-octylphenyl ether, and mixtures thereof.

26. The method of claim 25, wherein the surfactant is sorbitan monooleate.

27. The method of claim 15, wherein the second organic phase comprises an emulsion.

28. The method of claim 27, wherein the emulsion comprises water and the cross-linker.

29. The method of claim 15, wherein the cross-linker is selected from dithiothreitol, octanedithiol, and mixtures thereof.

30. The method of claim 29, wherein the cross-linker is dithiothreitol (DTT).

31. A method of detecting the presence or absence of an analyte, comprising:
obtaining a sample;
incubating one or more microcapsules of claim 1 in the sample;
retrieving the one or more microcapsules from the sample; and
imaging the one or more microcapsules to detect the presence or absence of the analyte.

32. The method of claim 31, wherein the sample is whole blood.

33. The method claim 31, wherein the analyte is a cytokine.

34. The method of claim 31, further comprising quantifying an amount of the analyte in the sample.
